(19) United States
(12) Patent Application Publication
(10) Pub. No.: US 2025/0113728 A1
(43) Pub. Date: Apr. 3, 2025

(54) MATERIAL FOR PHOTOELECTRIC CONVERSION ELEMENT FOR IMAGING, AND PHOTOELECTRIC CONVERSION ELEMENT

(71) Applicant: NIPPON STEEL CHEMICAL & MATERIAL CO., LTD., Tokyo (JP)

(72) Inventors: Munetomo INOUE, Tokyo (JP); Kentaro HAYASHI, Tokyo (JP)

(73) Assignee: NIPPON STEEL CHEMICAL & MATERIAL CO., LTD., Tokyo (JP)

(21) Appl. No.: 18/714,407

(22) PCT Filed: Jan. 12, 2023
(86) PCT No.: PCT/JP2023/000674

§ 371 Date: May 29, 2024

(30) Foreign Application Priority Data
Jan. 21, 2022 (JP) 2022-007814

(51) Int. Cl. H10K 85/60; C07D 333/10; C07D 407/10; C07D 409/10; H10K 30/30

(52) U.S. Cl. CPC H10K 85/655; C07D 333/10; C07D 407/10; C07D 409/10; H10K 85/622; H10K 85/626; H10K 85/6572; H10K 85/6574; H10K 30/353

(57) ABSTRACT

Provided is a material for a photoelectric conversion device for imaging that can achieve higher sensitivity and higher resolution.

A material for a photoelectric conversion device for imaging, the material including a thiophene derivative represented by the following general formula (1). A and B independently represents hydrogen, an aromatic hydrocarbon group having 6 to 30 carbon atoms, or the like, and at least one of A or B has a condensed-ring structure of the general formula (2) or (3). L¹, L², L³, L⁴, L⁵, and L⁶ independently represent an aromatic hydrocarbon group having 6 to 30 carbon atoms or the like; T represents a thiophene ring. “a” represents an integer of 1 to 4; “m”, “o”, “p”, “q”, “r”, and “s” each independently represent an integer of 0 or 1; and “n” and “t” independently represent 1 or 2. “*” represents a bonding position at which the formula (2) or (3) is substituted. X represents N—Ra, O, S, or C—(Rb)₂, and Ra and Rb independently represent an aromatic hydrocarbon group having 6 to 30 carbon atoms or the like.

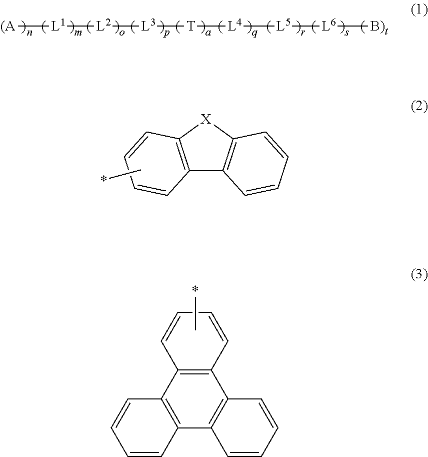

### TECHNICAL FIELD

The present invention relates to a material for a photoelectric conversion element (device) and a photoelectric conversion device using the same, and particularly to a material for a photoelectric conversion device useful for an imaging device.

In recent years, development of an organic electronic device using a thin film formed with an organic semiconductor is in progress. Examples thereof include an electroluminescent device, a solar cell, a transistor device, and a photoelectric conversion device. In particular, development of an organic EL device, which is an electroluminescent device with an organic substance, is most advanced among them. The applications for smartphones, TV and the like are in progress, and development for a purpose of further higher functionality is continuously conducted.

On the photoelectric conversion device, a device using a P-N junction of an inorganic semiconductor, such as silicon, has been conventionally developed and practically used, and made are investigations for high functionalization of a digital camera and a camera for a smartphone and investigation for application for a monitoring camera, a sensor for an automobile, and the like. However, problems for these various uses include improving sensitivity and micronizing a pixel (improving resolution). For the photoelectric conversion device using an inorganic semiconductor, a mainly adopted method for obtaining a color image is disposing color filters corresponding to RGB, which are the three primary colors of light, on a light receiving part of the photoelectric conversion device. This method has problems in terms of utilization efficiency of an incident light and resolution, because the method disposes the RGB color filters on a plane (Non Patent Literature 1 and 2).

As a solution for such problems of the photoelectric conversion device, a photoelectric conversion device using an organic semiconductor instead of the inorganic semiconductor is developed (Non Patent Literature 1 and 2). This utilizes an ability to selectively absorb only light having a specific wavelength region with high sensitivity that the organic semiconductor has, and proposed is stacking photoelectric conversion devices composed of organic semiconductors corresponding to the three primary colors of light to solve the problem of improving the sensitivity and improving the resolution. A device in which a photoelectric conversion device composed of the organic semiconductor and a photoelectric conversion device composed of the inorganic semiconductor are stacked is also proposed (Non Patent Literature 3).

Here, the photoelectric conversion device using the organic semiconductor is a device having a photoelectric conversion layer composed of a thin film of the organic semiconductor between two electrodes, wherein a hole blocking layer and/or an electron blocking layer is disposed between the photoelectric conversion layer and the two electrodes, as necessary. In the photoelectric conversion device, light having a desired wavelength is absorbed in the photoelectric conversion layer to generate an exciton, and then charge separation of the exciton generates a hole and an electron. Thereafter, the hole and the electron move toward each electrode to convert the light into an electric signal. For a purpose of accelerating this process, a method of applying a bias voltage between both the electrodes is commonly used, but one of objects is reducing a leakage current from both the electrodes generated by applying the bias voltage. Accordingly, it can be mentioned that controlling the move of the hole and the electron in the photoelectric conversion device is a key to exhibit characteristic of the photoelectric conversion device.

The organic semiconductor used for each layer of the photoelectric conversion device can be classified into a P-type organic semiconductor and an N-type organic semiconductor. The P-type organic semiconductor is used as a hole transport material, and the N-type organic semiconductor is used as an electron transport material. To control the move of the hole and the electron in the aforementioned photoelectric conversion device, made are various developments of an organic semiconductor having appropriate physical properties such as hole mobility, electron mobility, an energy value of a highest occupied molecular orbital (HOMO), and an energy value of a lowest unoccupied molecular orbital (LUMO). However, the organic semiconductor still has insufficient characteristics, and has not been utilized in commercial practice.

Patent literature 1 proposes a device using a carbazole derivative for the electron blocking layer disposed between the photoelectric conversion layer and the electrode.

Patent literature 2 to 5 proposes a photoelectric conversion device using a thiophene derivative having a specific substituent.

### CITATION LIST

### Patent Literature

### Patent Literature 1

JP 2011-228614 (A)

### Patent Literature 2

JP 2010-93100 (A)

### Patent Literature 3

JP 2018-85499 (A)

### Patent Literature 4

WO2019/058995

### Patent Literature 5

WO2021/221108

### Non Patent Literature

### Non Patent Literature 1

NHK Science & Technology Research Laboratories R&D No. 132, pp. 4-11 (2012.3)

### Non Patent Literature 2

NHK Science & Technology Research Laboratories R&D No. 174, pp. 4-17 (2019.3)

### Non Patent Literature 3

2019 IEEE International Electron Devices Meeting (IEDM), pp. 16.6.1-16.6.4 (2019)

### SUMMARY OF INVENTION

### Technical Problem

In the use of the photoelectric conversion device for imaging for highly functionalizing a digital camera and a camera for a smartphone and for application for a monitoring camera, a sensor for an automobile, and the like, challenges are further higher sensitivity and higher resolution. In view of such a circumstance, an object of the present invention is to provide a material that achieves higher sensitivity and higher resolution of the photoelectric conversion device for imaging, and a photoelectric conversion device for imaging using the same.

### Solution to Problem

The present inventors have made intensive investigation, and consequently found that using a thiophene compound having a specific substituent efficiently proceeds a process of generating a hole and an electron by charge separation of an exciton in a photoelectric conversion layer, and a process of moving of the hole and the electron in the photoelectric conversion device. This finding has led to the completion of the present invention.

The present invention is a material for a photoelectric conversion device for imaging, the material comprising a thiophene derivative represented by the following general formula (1) or (1T).

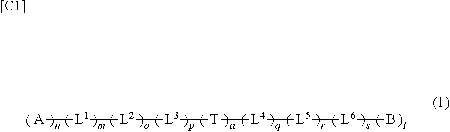

Here, A and B each independently represent hydrogen, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to four of these aromatic groups are linked, and at least one of A or B has a condensed-ring structure having 12 or more carbon atoms represented by the following general formula (2) or (3).

L¹, L², L³, L⁴, L⁵, and L⁶ each independently represent a substituted or unsubstituted aromatic hydrocarbon group having 6 to 30 carbon atoms, or a substituted or unsubstituted aromatic heterocyclic group having 3 to 18 carbon atoms.

T represents a substituted or unsubstituted thiophene ring. “a” represents the number of repetition and represents an integer of 1 to 4; “m”, “o”, “p”, “q”, “r”, and “s” represent the number of linkage and each independently represent an integer of 0 or 1, and “n” and “t” represent the number of substitution and each independently represent 1 or 2. “a” preferably represents 1 to 3.

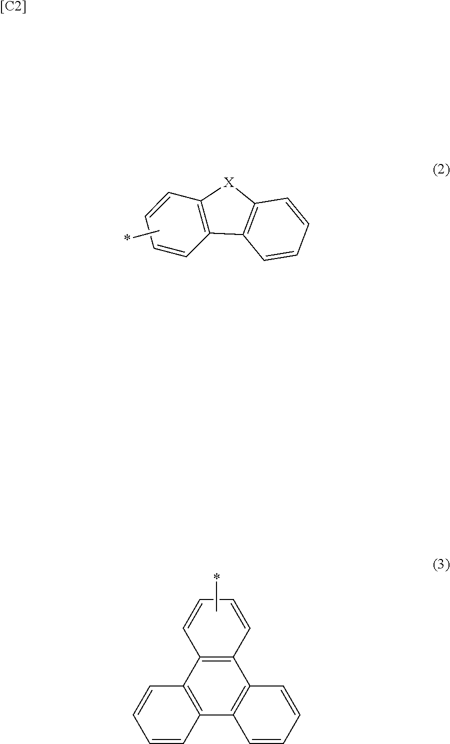

Here, “*” represents a bonding position at which the formula (2) or (3) is substituted, the condensed-ring structures represented by the formulae (2) and (3) optionally have a substituent, and the formula (2) is optionally condensed with the substituent to form a condensed ring.

X represents N—Ra, O, S, or C—(Rb)₂, Ra and Rb each independently represent a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to four of these aromatic groups are linked, and Ra and Rb are optionally bonded to the formula (2) to form a condensed ring.

The general formula (1) is preferably represented by the following general formula (1T):

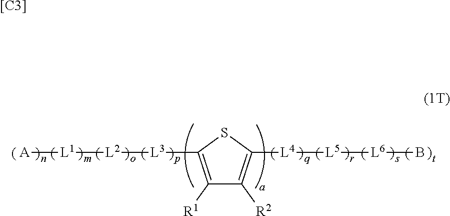

wherein R¹ and R² each independently represent hydrogen, a substituted or unsubstituted aromatic hydrocarbon group having 6 to 30 carbon atoms, or a substituted or unsubstituted aromatic heterocyclic group having 3 to 18 carbon atoms; and A, B, L¹ to L⁶, “a”, and “n” to “t” are as defined for the general formula (1).

In the general formula (1) or the general formula (1T), at least one of A or B is preferably represented by the general formula (2), and X in the formula (2) preferably represents N—Ra or O.

In the general formula (1) or the general formula (1T), at least one of A or B is preferably represented by the following general formula (4), and more preferably both A and B are represented by the general formula (4):

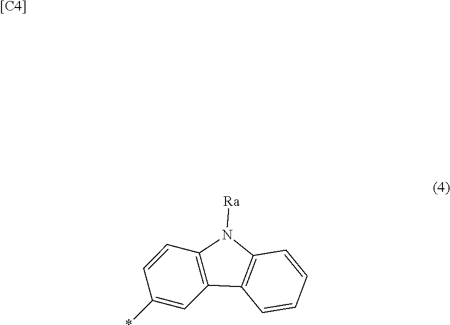

“*” is as defined for the formula (2); the formula (4) optionally has a substituent; Ra is as defined for the formula (2), and optionally further forms a condensed ring with the formula (4) itself or the substituent in the formula (4); and the formula (4) is optionally condensed with the substituent in the formula (4) itself to form a condensed ring.

In the general formula (1) or the general formula (1T), at least one of A or B is preferably represented by the following formula (5), and more preferably both A and B are represented by the following formula (5):

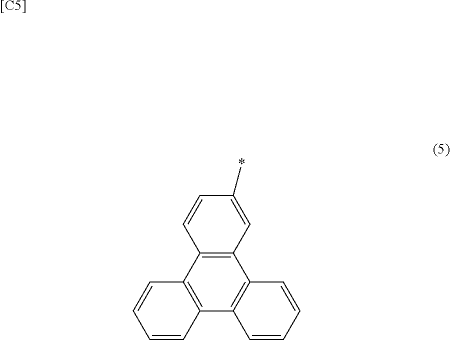

wherein “*” is as defined for the formula (3); and the formula (5) optionally has a substituent.

In the general formula (1), at least one of L¹, L², L³, L⁴, L⁵, or L⁶ is preferably represented by the following formula (6), and at least one of L¹, L², L³, L⁴, L⁵, or L⁶ is more preferably represented by the following general formulae (6a) to (6d):

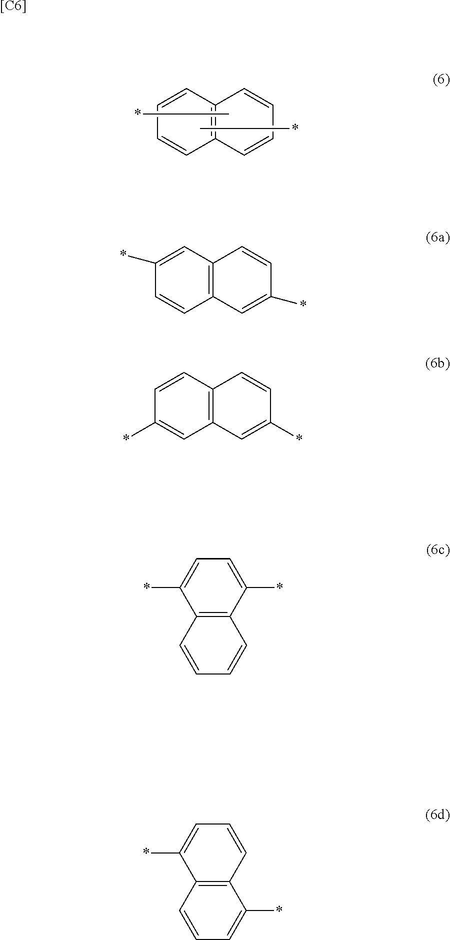

wherein “*” represents a bonding position to be substituted with A, B, L¹, L², L³, L⁴, L⁵, L⁶, or T; and the formula (6) and the formulae (6a) to (6d) optionally have a substituent.

In the material for a photoelectric conversion device, preferably satisfied is any one of requirements that: an energy level of highest occupied molecular orbital (HOMO) obtained by structural optimization calculation with density functional calculation B3LYP/6-31G (d) is −4.5 eV or lower; an energy level of lowest unoccupied molecular orbital (LUMO) obtained by the structural optimization calculation is −2.5 eV or higher; the material has a hole mobility of 1×10⁻⁶ cm²/Vs or more; or the material is amorphous.

The material for a photoelectric conversion device may be used as a hole transport material of a photoelectric conversion device for imaging.

The present invention is a photoelectric conversion device for imaging, comprising a photoelectric conversion layer and an electron blocking layer between two electrodes, wherein at least one layer of the photoelectric conversion layer or the electron blocking layer contains the above material for a photoelectric conversion device.

The above material for a photoelectric conversion device is preferably contained in an electron blocking layer or a photoelectric conversion layer of the photoelectric conversion device, and in this case, preferably contained as a hole transport material. When the material for a photoelectric conversion device is contained in the electron blocking layer, the photoelectric conversion layer preferably contains an electron transport material or a fullerene derivative.

### Advantageous Effect of Invention

The material for a photoelectric conversion device for imaging of the present invention can achieve appropriate move of the hole and the electron in the photoelectric conversion device, and consequently enables to reduce a leakage current generated by applying a bias voltage during the conversion of light into electric energy. As a result, a photoelectric conversion device that achieves a low dark current value and a high contrast ratio can be obtained. The material of the present invention is useful as a material for a photoelectric conversion device for a photoelectric-converting film-stacked imaging device.

### BRIEF DESCRIPTION OF DRAWING

FIG. 1 is a sectional schematic view illustrating a structure example of a photoelectric conversion device for imaging.

### DESCRIPTION OF EMBODIMENTS

The photoelectric conversion device for imaging of the present invention has at least one organic layer between two electrodes. This organic layer contains the material for a photoelectric conversion device for imaging represented by the general formula (1) or (1T).

Hereinafter, the material for a photoelectric conversion device for imaging represented by the general formula (1) or (1T) is also referred to as a material for a photoelectric conversion device, a material of the present invention, or the compound represented by the general formula (1) or (1T).

The compound represented by the general formula (1) or (1T) will be described below.

In the general formula (1), T represents a thiophene ring, and is bonded at any position to adjacent A, B, L¹, L², L³, L⁴, L⁵ or L⁶ (“n” and “t” in the general formula (1) represents the number of substitution, and each independently represent 1 or 2). This thiophene ring may have a substituent.

In the general formula (1) or (1T), “a” represents the number of repetition and represents an integer of 1 to 4, “m”, “o”, “p”, “q”, “r”, and “s” represent the number of linkage and each independently represent an integer of 0 or 1, and “n” and “t” represent the number of substitution and each independently represent 1 or 2. “a” preferably represents an integer of 1 to 3, and more preferably represents 1 or 2. n+m+o+p+q+r+s+t is preferably an integer of 2 to 7, and more preferably 3 to 5.

A and B each independently represent hydrogen, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to four of these aromatic groups are linked, and preferably represent hydrogen, a substituted or unsubstituted aromatic hydrocarbon group having 6 to 18 carbon atoms, a substituted or unsubstituted aromatic heterocyclic group having 3 to 12 carbon atoms, or a substituted or unsubstituted linked aromatic group in which two to four of these aromatic groups are linked. At least one of A or B has a condensed-ring structure having 12 or more carbon atoms selected from any one of the formulae (2) to (5). When A and B represent the linked aromatic groups, the formulae (2) to (5) may be terminals or may be contained in the middle of the linked aromatic group, but preferably contained at the terminal.

Examples of the unsubstituted aromatic hydrocarbon compound having 6 to 30 carbon atoms include a group generated from: monocyclic aromatic hydrocarbons, such as benzene and biphenyl; bicyclic aromatic hydrocarbons, such as naphthalene; tricyclic aromatic hydrocarbons, such as indacene, biphenylene, phenalene, anthracene, phenanthrene, and fluorene; tetracyclic aromatic hydrocarbons, such as fluoranthene, acephenanthrylene, aceanthrylene, triphenylene, pyrene, chrysene, tetraphene, tetracene, and pleiadene; pentacyclic aromatic hydrocarbons, such as picene, perylene, pentaphene, pentacene, tetraphenylene, and naphthoanthracene; and the like. The unsubstituted aromatic hydrocarbon compound is preferably a group generated from benzene, naphthalene, anthracene, phenanthrene, triphenylene, or pyrene.

Examples of the unsubstituted aromatic heterocyclic group having 3 to 18 carbon atoms include a group generated from: nitrogen-containing aromatic compounds having a pyrrole ring, such as pyrrole, pyrrolopyrrole, indole, isoindole, pyrroloisoindole, and carboline; thiophene, benzothiophene, dibenzothiophene, furan, benzofuran, dibenzofuran, carbazole, indolocarbazole, pyridine, pyrimidine, triazine, quinoline, isoquinoline, quinazoline, quinoxaline, or the like. Note that A and B exclude thiophene. Preferable examples of A and B include: a group generated from benzene, naphthalene, anthracene, phenanthrene, pyrene, dibenzothiophene, furan, dibenzofuran, carbazole, quinoline, isoquinoline, quinazoline, or quinoxaline; the structure represented by the formulae (2) to (5); or a substituted or unsubstituted linked aromatic group in which two to four of these aromatic groups are linked.

Examples of the substituent herein include a deuterium, a cyano group, an alkyl group having 1 to 20 carbon atoms, a diarylamino group having 12 to 30 carbon atoms, an unsubstituted arylheteroarylamino group having 12 to 30 carbon atoms, or an unsubstituted diheteroarylamino group having 12 to 30 carbon atoms.

When the substituent is the unsubstituted diarylamino group having 12 to 30 carbon atoms, the unsubstituted arylheteroarylamino group having 12 to 30 carbon atoms, or the unsubstituted diheteroarylamino group having 12 to 30 carbon atoms, specific examples thereof include diphenylamino, dibiphenylamino, phenylbiphenylamino, naphthylphenylamino, dinaphthylamino, dianthranilamino, diphenanthrenylamino, dibenzofuranylphenylamino, dibenzofuranylbiphenylamino, or bisdibenzofuranylamino. Preferable examples thereof include diphenylamino, dibiphenylamino, phenylbiphenylamino, naphthylphenylamino, dinaphthylamino, dibenzofuranylphenylamino, dibenzofuranylbiphenylamino, and bisdibenzofuranylamino. More preferable examples thereof include diphenylamino, phenylbiphenylamino, dibenzofuranylphenylamino, or dibenzofuranylbiphenylamino. The aryl group constituting the above amino groups is preferably an aryl group having 6 to 18 carbon atoms, and the heteroaryl group is preferably a heteroaryl group having 6 to 15 carbon atoms. The number of carbon atoms of these amino groups is preferably 12 to 24. A heteroatom in the heteroaryl group is preferably N, S, or O. When the diarylamino group, the arylheteroarylamino group, or the diheteroarylamino group is the substituent of the formula (2) or (4), the substituent is optionally condensed with the formula (2) or (4) to form a condensed-ring structure, and examples thereof include compounds such as example compounds (51) and (52).

When the substituent is an alkyl group having 1 to 20 carbon atoms, the alkyl group may be any of a linear, branched, or cyclic alkyl group, and is preferably a linear, branched, or cyclic alkyl group having 1 to 10 carbon atoms. Specific examples thereof include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a tert-butyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, a 4-butylcyclohexyl group, and a 4-dodecylcyclohexyl group.

In the present invention, the substituent is bonded to a carbon atom or a heteroatom that constitute the aromatic ring. Note that, when T has the substituent, the substituent is R¹ and R².

In the formulae (2) to (5), “*” represents a bonding position at which the structure of each of the formulae is substituted, and the formulae (2) to (5) optionally have a substituent.

Preferable aspects of the formula (2) include the aspect represented by the formula (4), and preferable aspects of the formula (3) include the aspect represented by the formula (5).

Note that the formula (2) or the formula (4) is optionally condensed with the substituent to form a condensed ring.

X represents N—Ra, O, S, or C—(Rb)₂, preferably N—Ra or O, and more preferably N—Ra.

Ra and Rb each independently represent a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to four of these aromatic groups are linked. Examples of the unsubstituted aromatic hydrocarbon compound having 6 to 30 carbon atoms and the unsubstituted aromatic heterocyclic group having 3 to 18 carbon atoms are same as the case described in the unsubstituted aromatic hydrocarbon compound having 6 to 30 carbon atoms and the unsubstituted aromatic heterocyclic group having 3 to 18 carbon atoms. Ra and Rb preferably represent benzene, naphthalene, anthracene, phenanthrene, triphenylene, pyrene, dibenzothiophene, dibenzofuran, carbazole, or a substituted or unsubstituted linked aromatic group in which two to four of these aromatic groups are linked.

Ra and Rb are optionally bonded to the formula (2) or the formula (4) to form a condensed ring. Specific examples in this case include example compounds (9), (10), (11), and (12).

L¹, L², L³, L⁴, L⁵, and L⁶ independently represent an aromatic group generated by removing two hydrogens from a substituted or unsubstituted aromatic hydrocarbon group having 6 to 30 carbon atoms or a substituted or unsubstituted aromatic heterocyclic group having 3 to 18 carbon atoms, and preferably represent an aromatic group generated by removing two hydrogens from a substituted or unsubstituted aromatic hydrocarbon group having 6 to 18 carbon atoms or a substituted or unsubstituted aromatic heterocyclic group having 3 to 12 carbon atoms. Note that at least one of L¹, L², L³, L⁴, L⁵, or L⁶ preferably represents the aromatic group represented by the formula (6), and more preferably represented by the formulae (6a) to (6d). L¹ to L⁶ do not represent thiophene.

Examples of the unsaturated aromatic hydrocarbon compound having 6 to 30 carbon atoms or the unsubstituted aromatic heterocyclic group having 3 to 18 carbon atoms are same as the case described in the unsaturated aromatic hydrocarbon compound having 6 to 30 carbon atoms except that thiophene is excluded. Examples thereof also preferably include the unsubstituted aromatic compound having a structure represented by the formula (6) or (6a) to (6d). This aromatic group may have a substituent. The substituent that the formulae (6) and (6a) to (6d) have are same as the substituent that the aromatic group represented by the formula A or B may have. This substituent is bonded to a carbon atom or a heteroatom that constitute the aromatic ring in the formulae (6) and (6a) to (6d).

In the formulae (6) and (6a) to (6d), “*” represents a bonding position to the adjacent A, B, L¹, L², L³, L⁴, L⁵, L⁶, or T.

The aromatic hydrocarbon group represented by the formulae (6) and (6a) to (6d) has two bonding points (or bonds; represented by “*”), and when having the substituent, the substituent may be bonded at any position other than “*”.

The general formula (1) is preferably represented by the general formula (1T), and R¹ and R² in the general formula (1T) each independently represent hydrogen, a substituted or unsubstituted aromatic hydrocarbon group having 6 to 30 carbon atoms, or a substituted or unsubstituted aromatic heterocyclic group having 3 to 18 carbon atoms. Preferable examples of this aromatic group are same as in the case represented by A or B. This aromatic group may have a substituent. This substituent is same as the substituent that the aromatic group represented by A or B may have. The substituent is bonded to a carbon atom or a heteroatom that constitute the aromatic ring.

Preferable specific examples of the material for a photoelectric conversion device represented by the general formula (1) or the general formula (1T) of the present invention will be described below, but the material is not limited thereto.

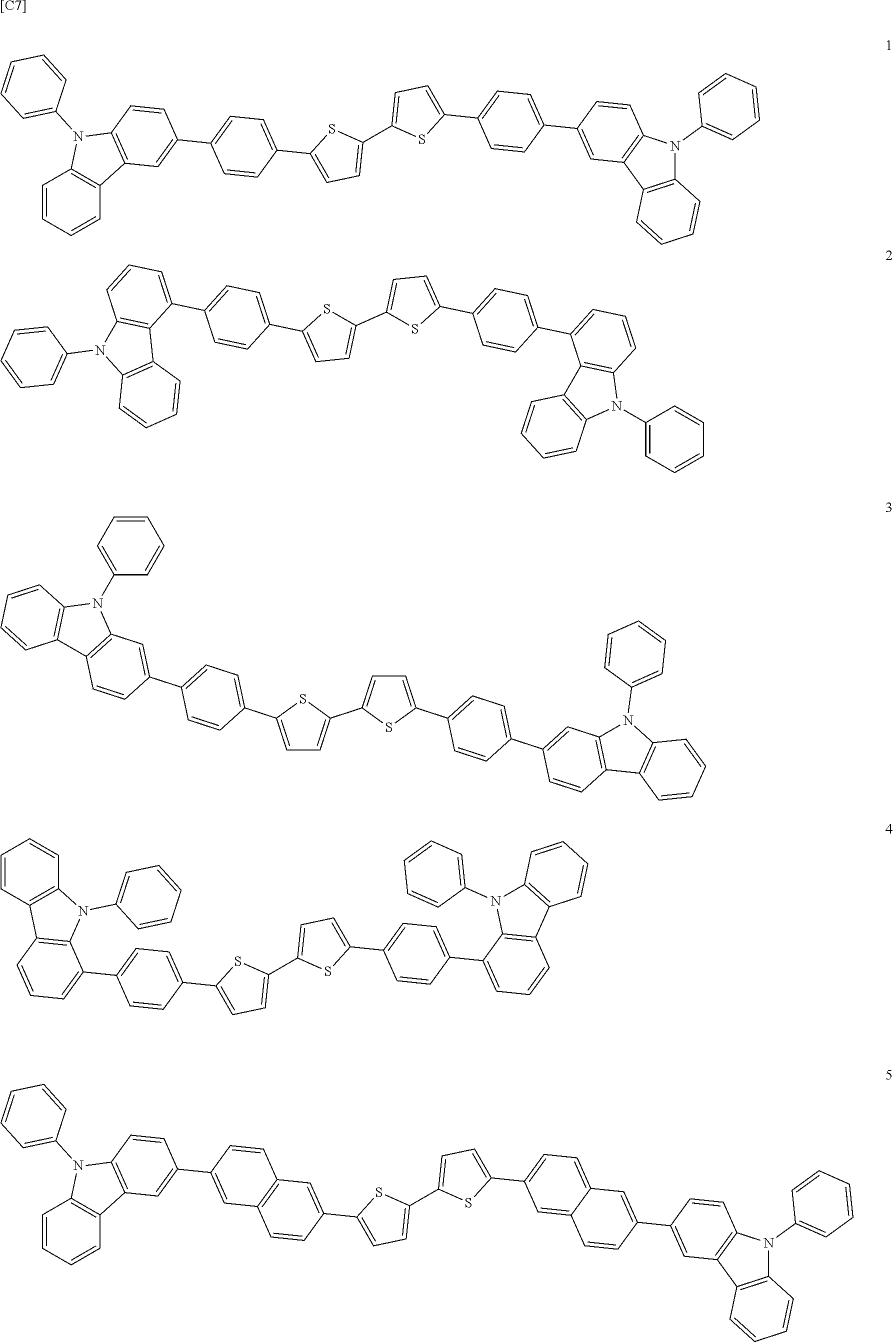

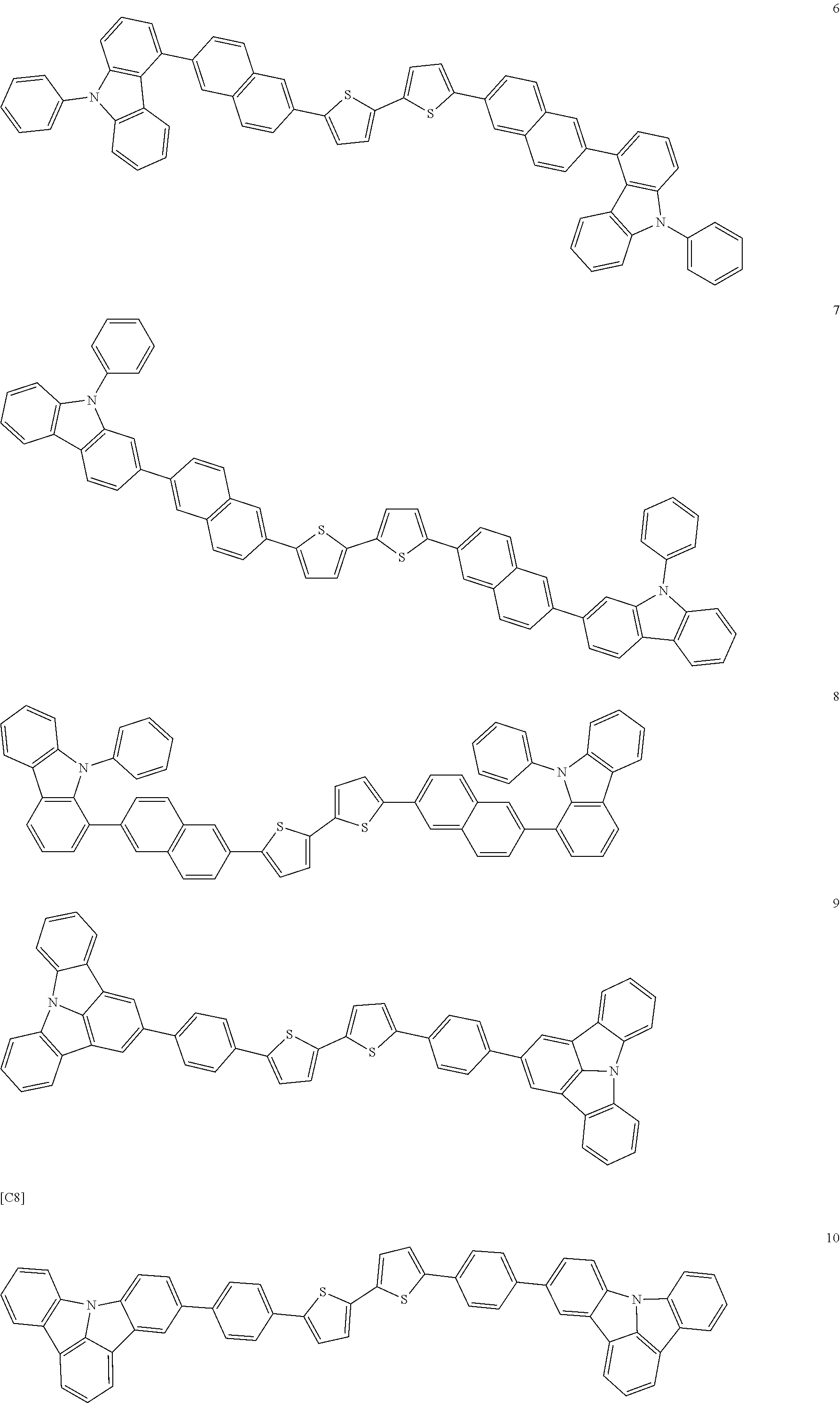

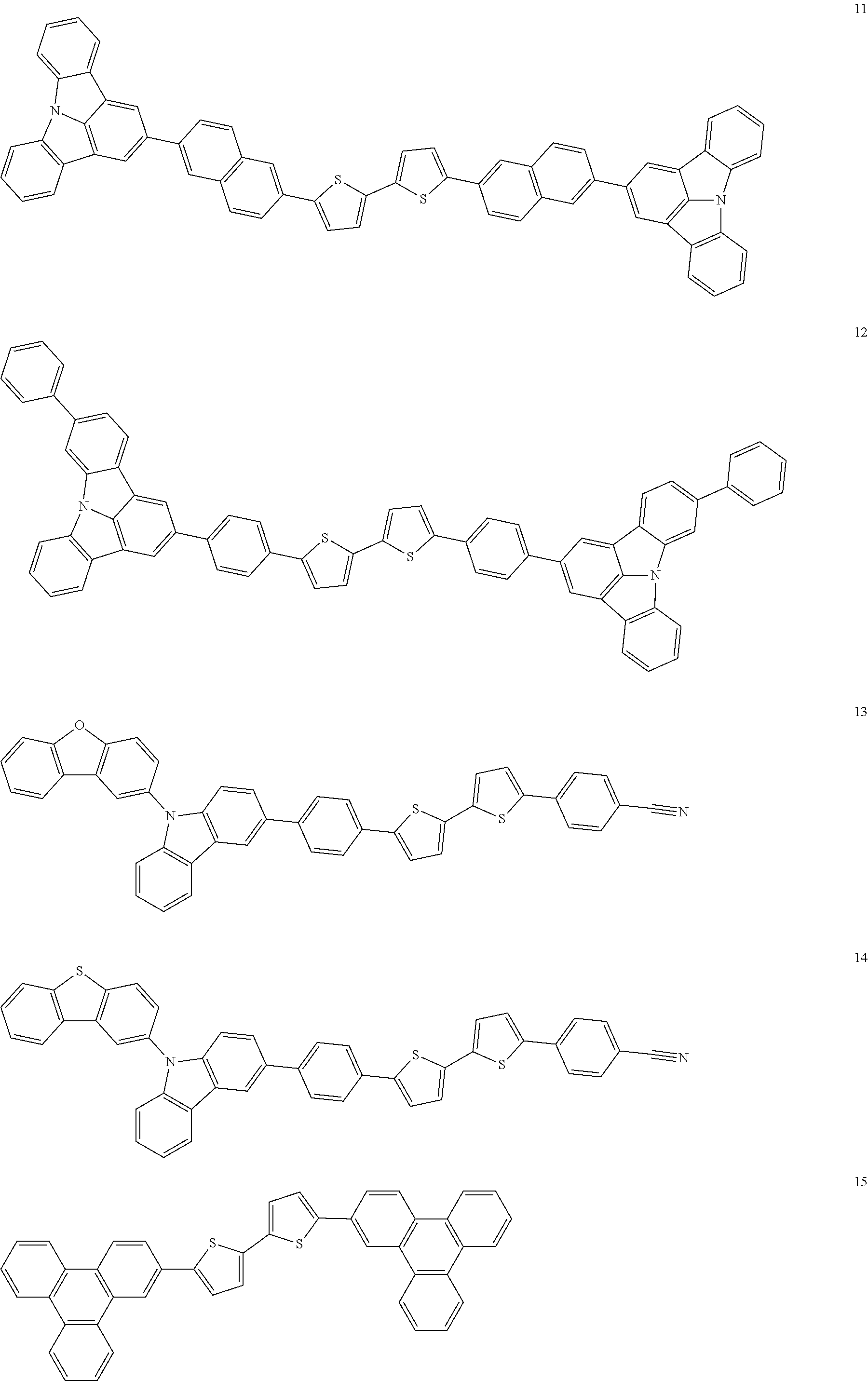

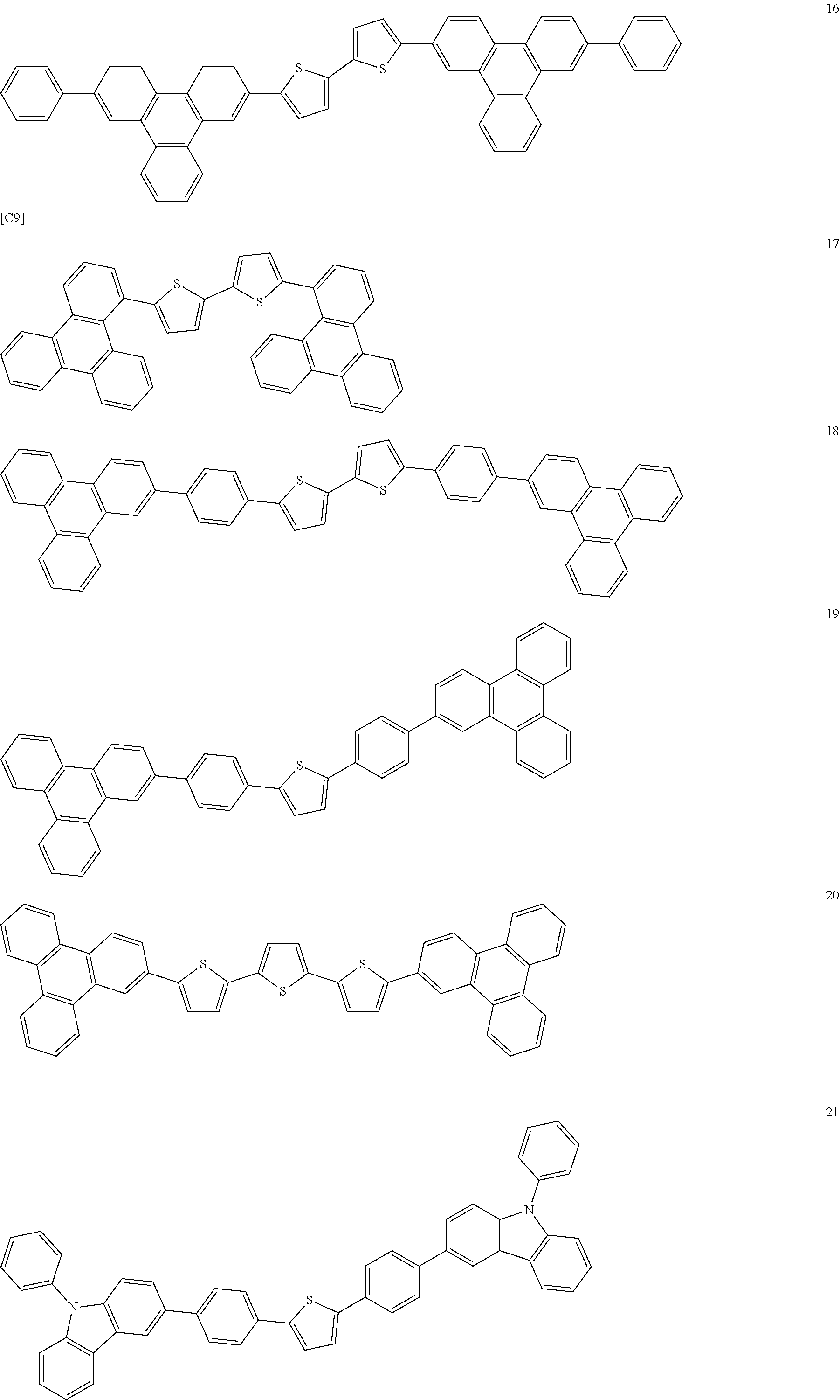

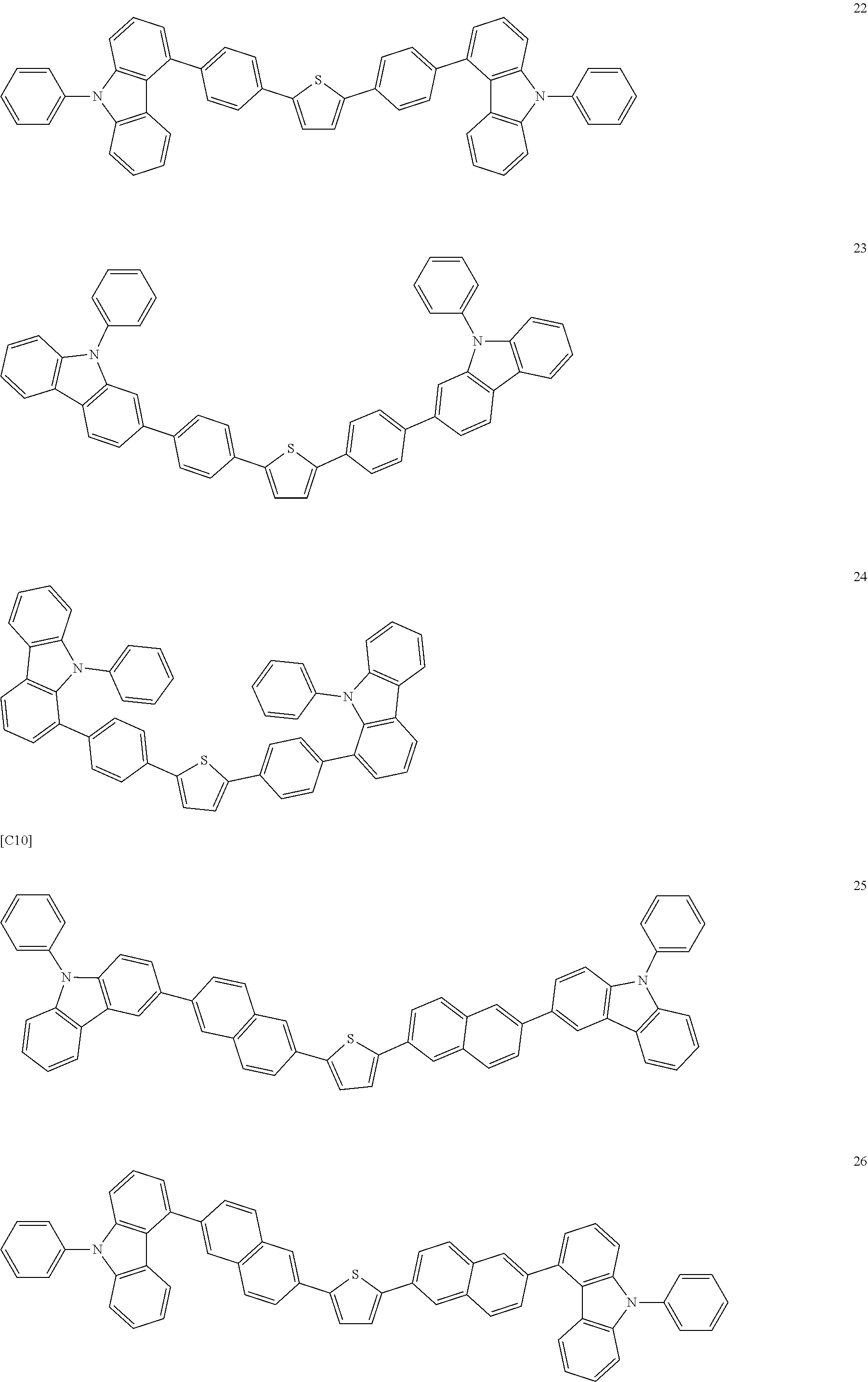

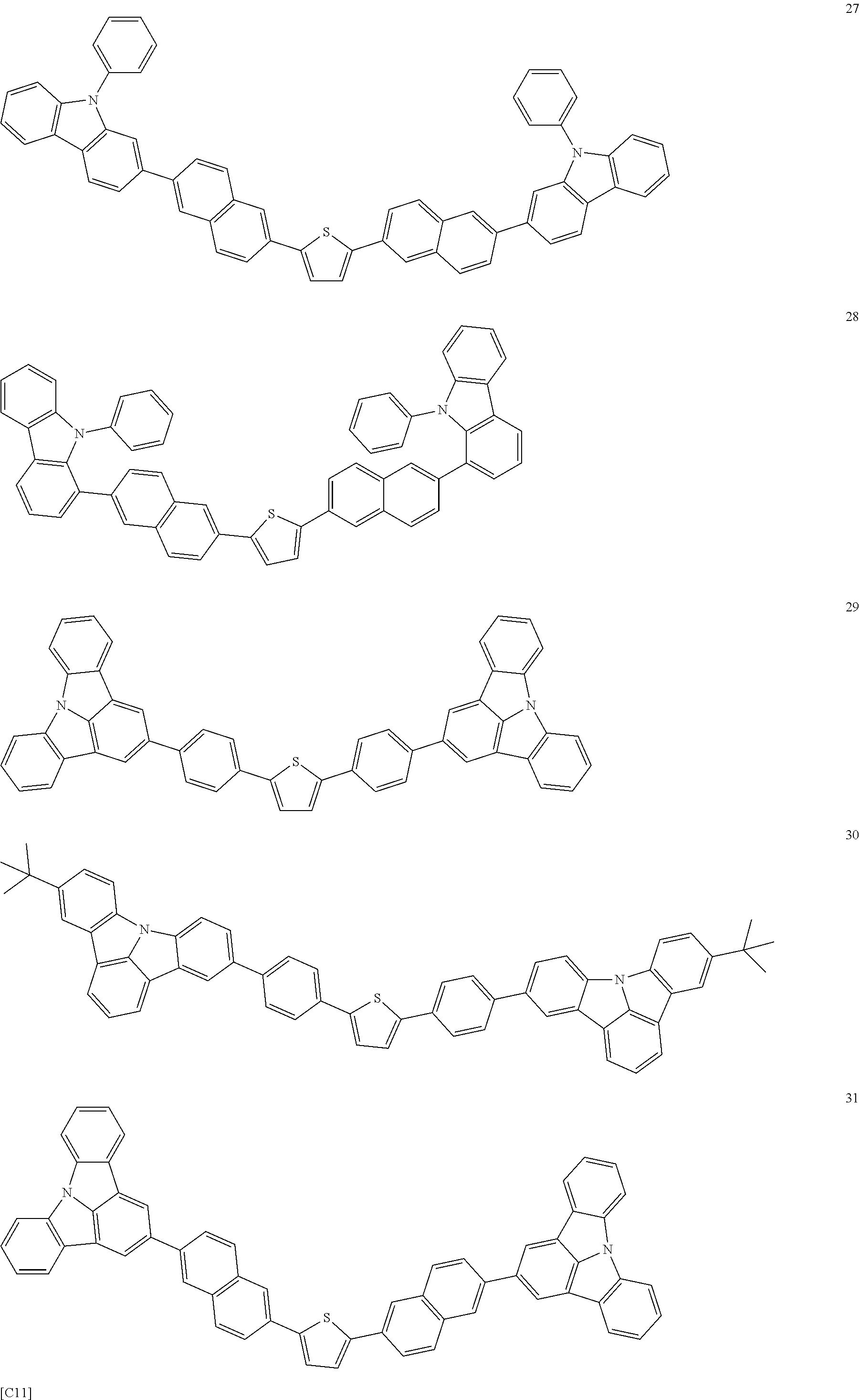

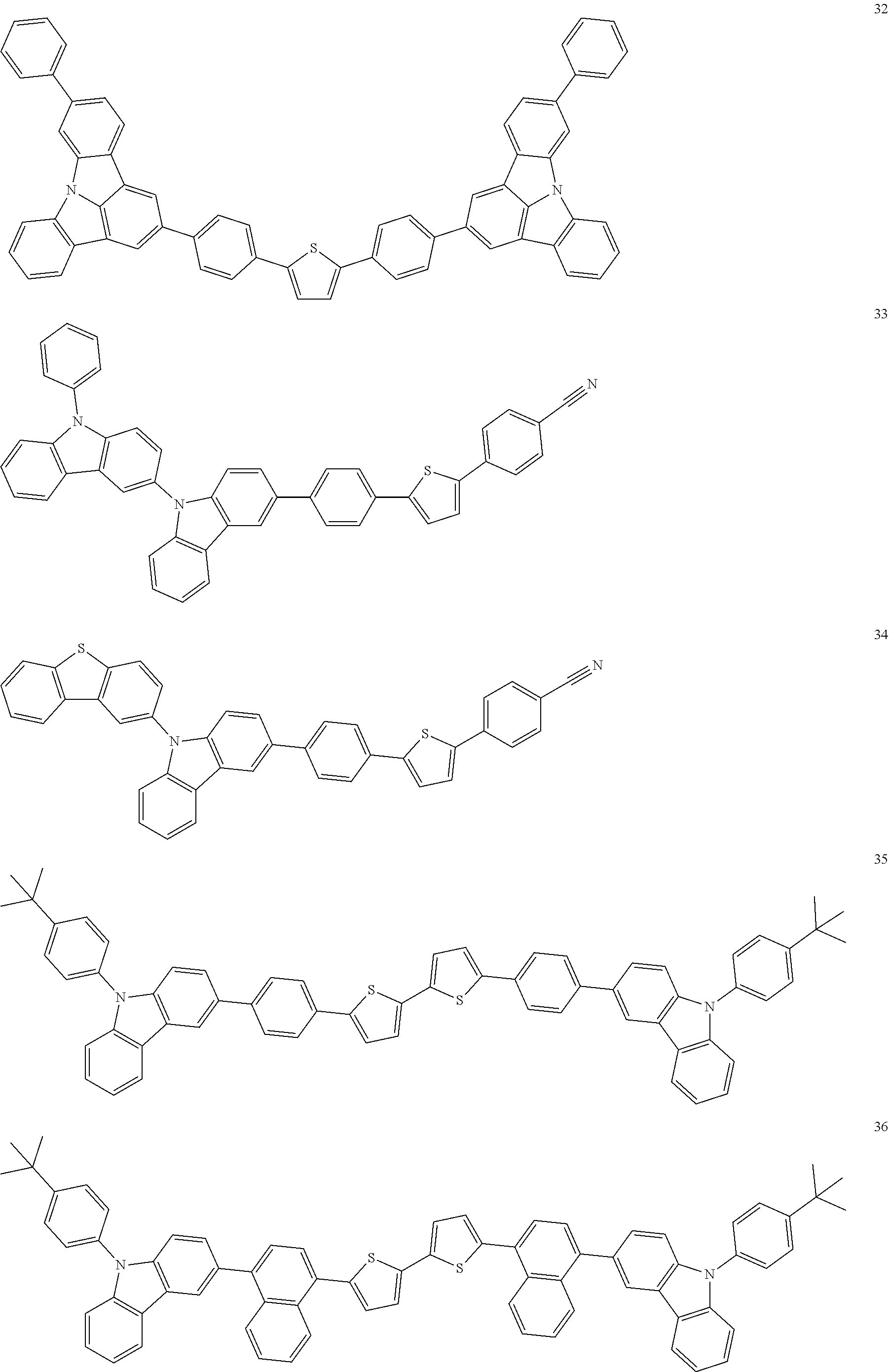

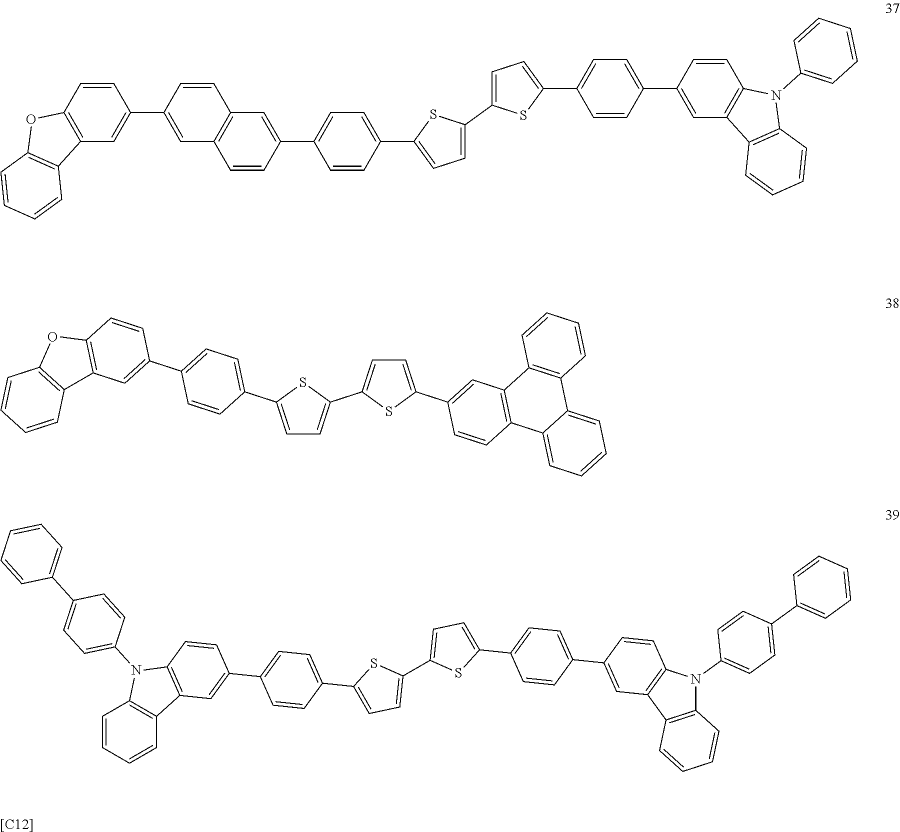

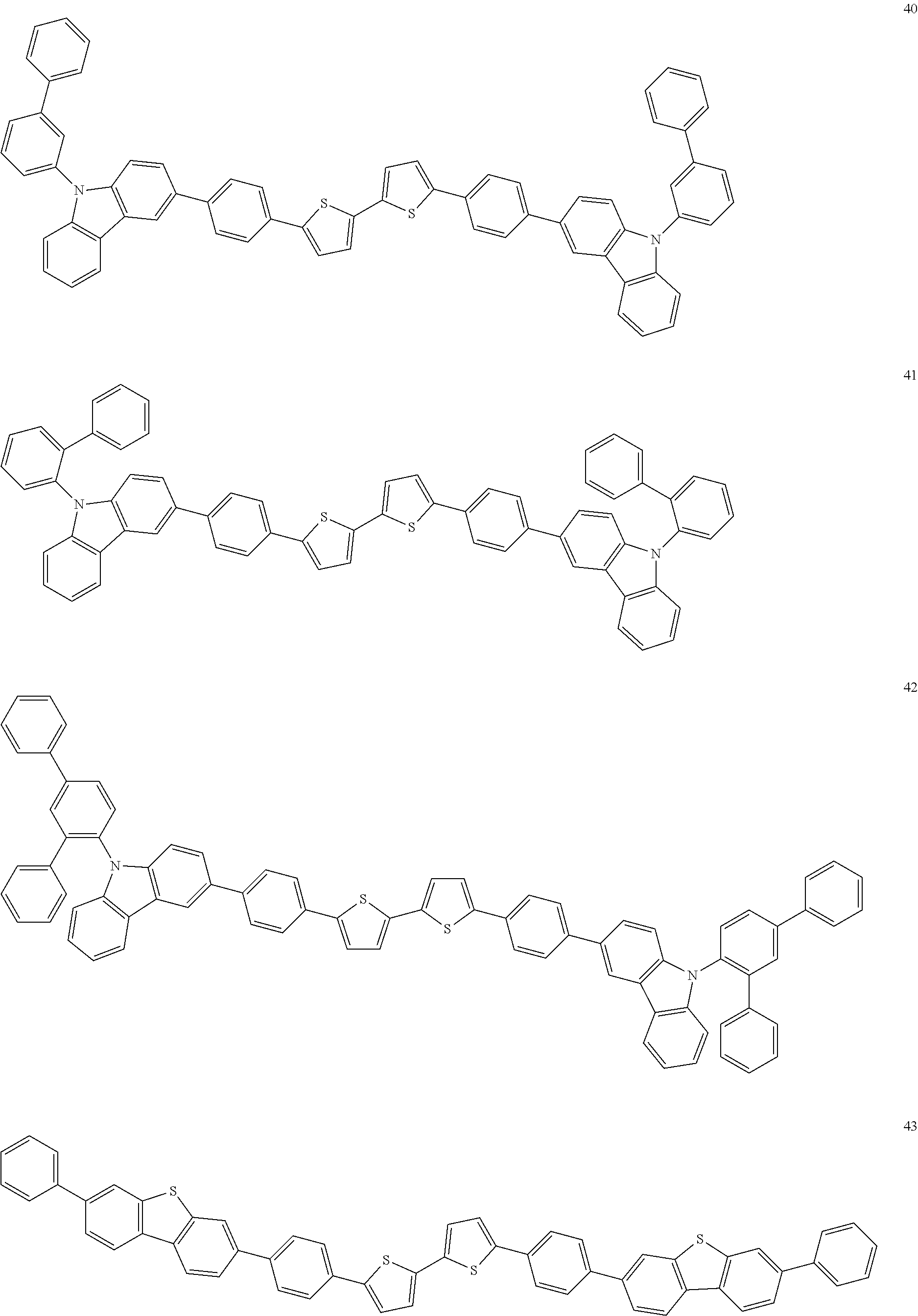

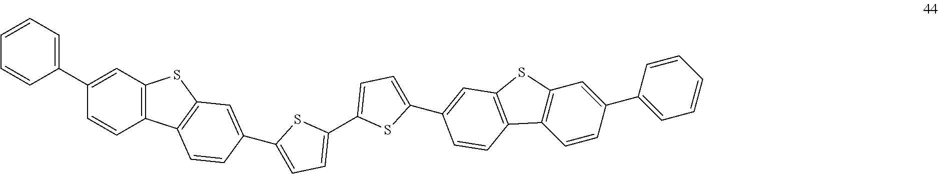

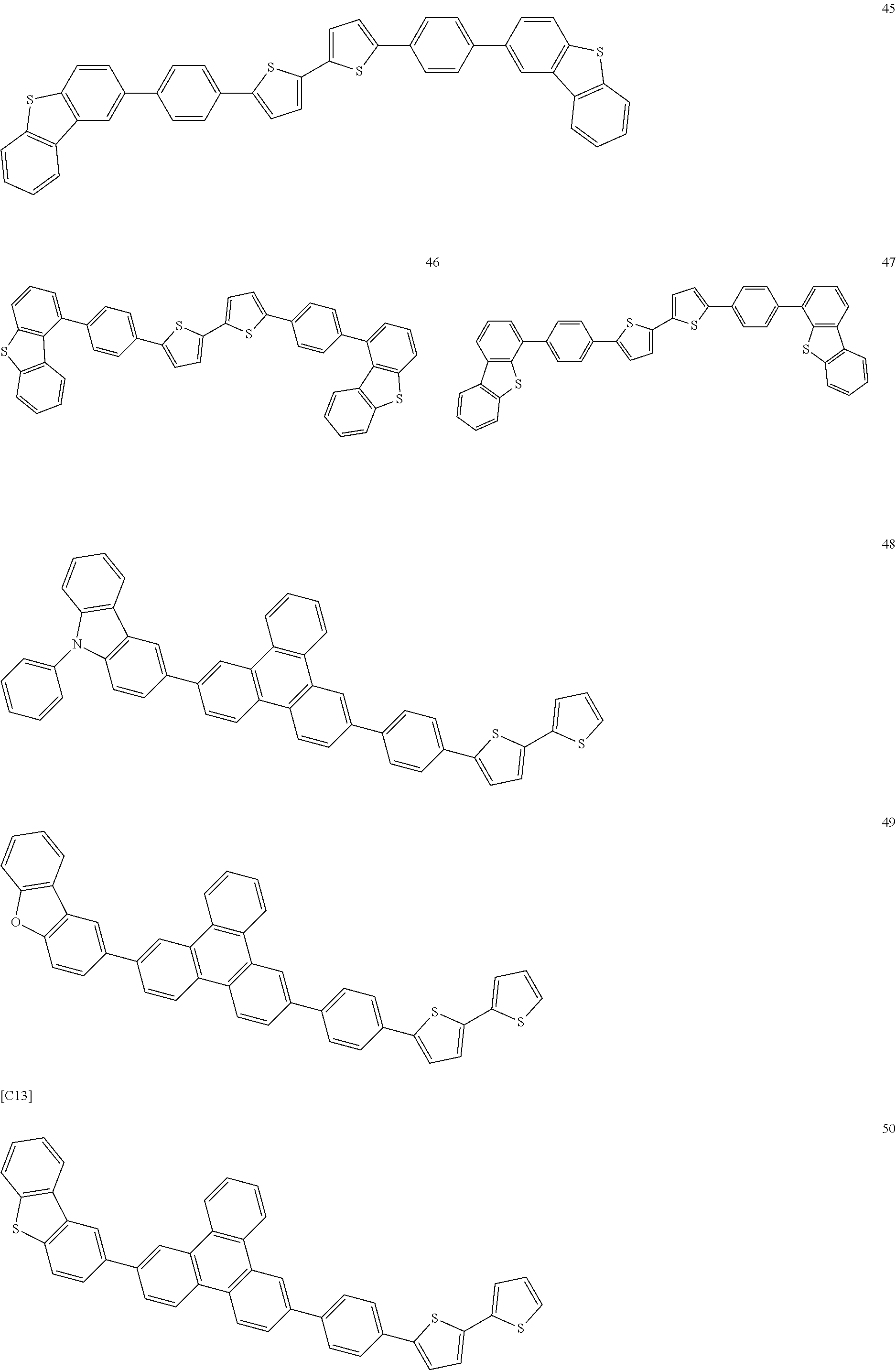

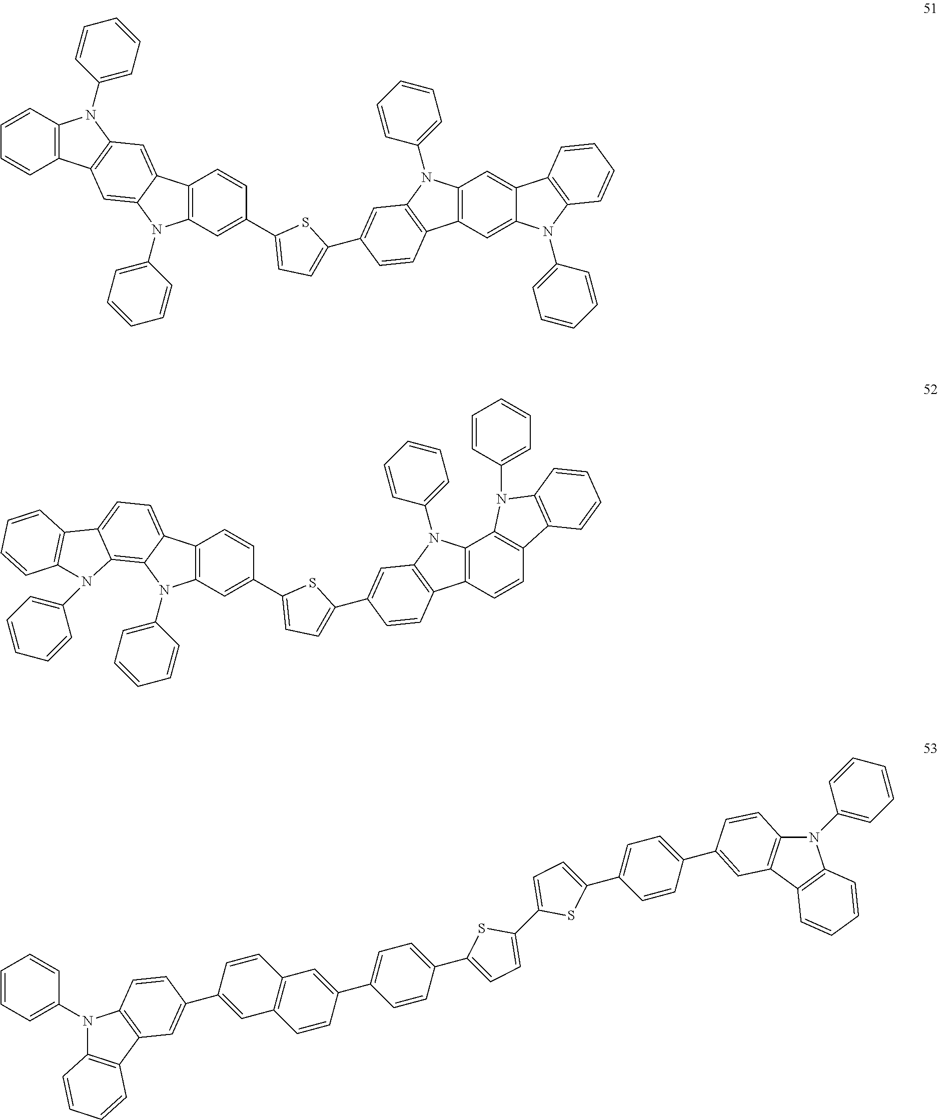

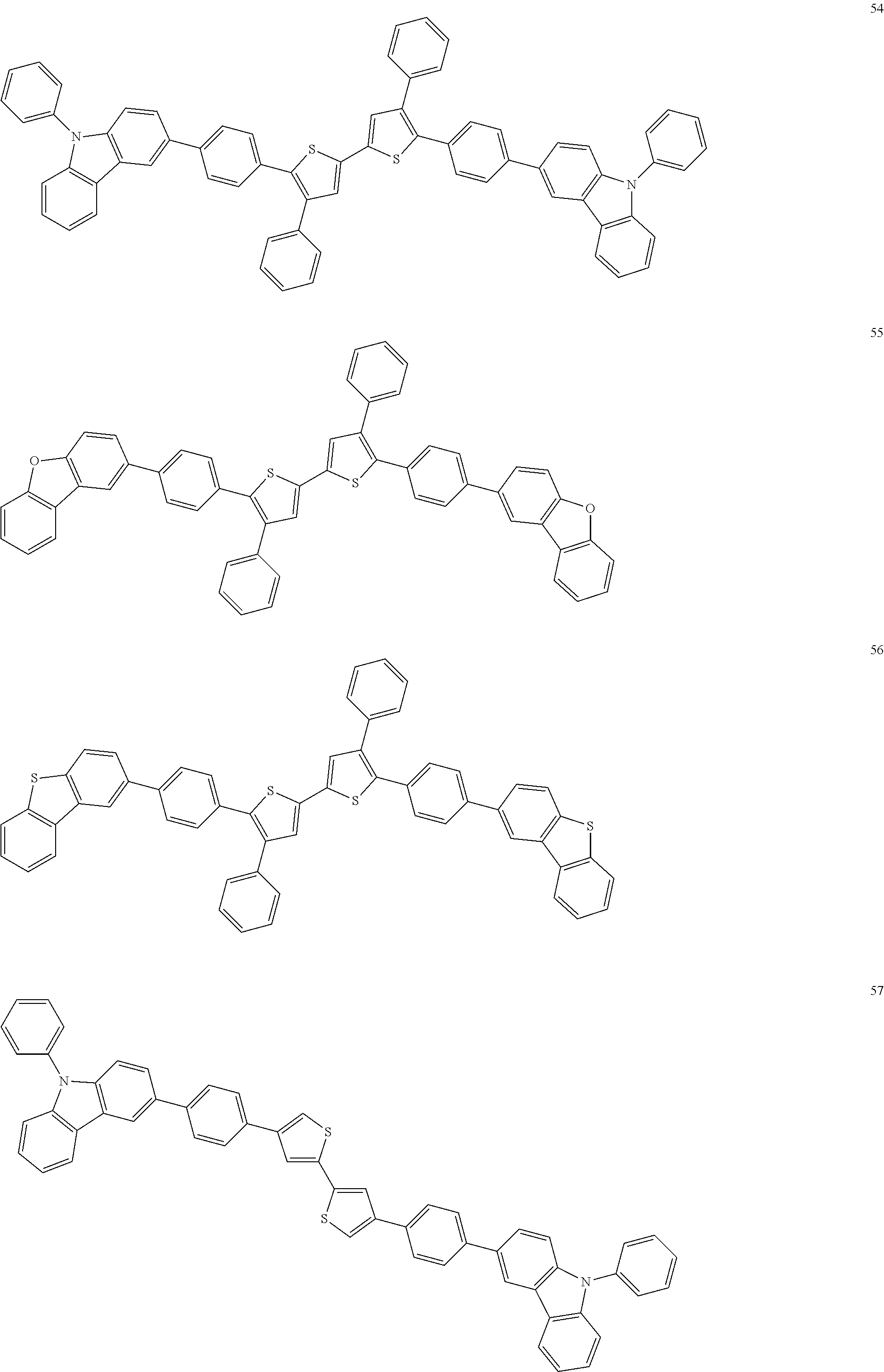

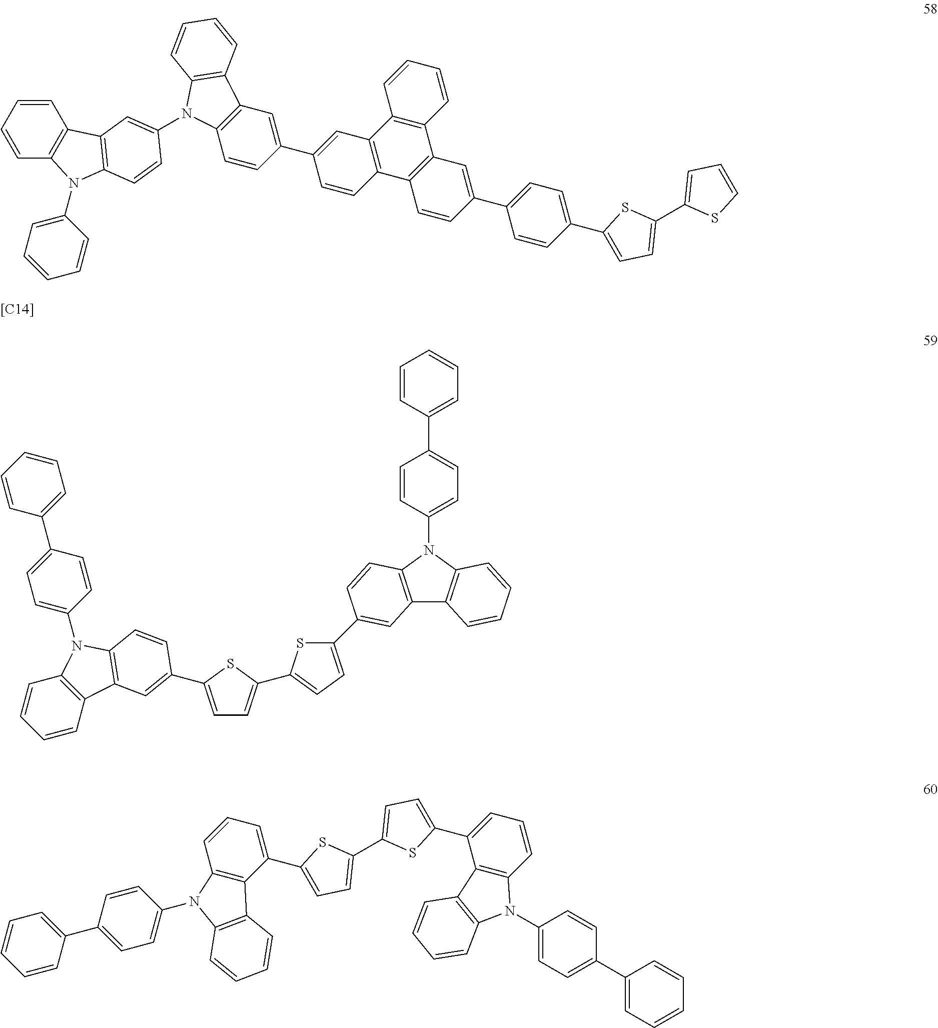

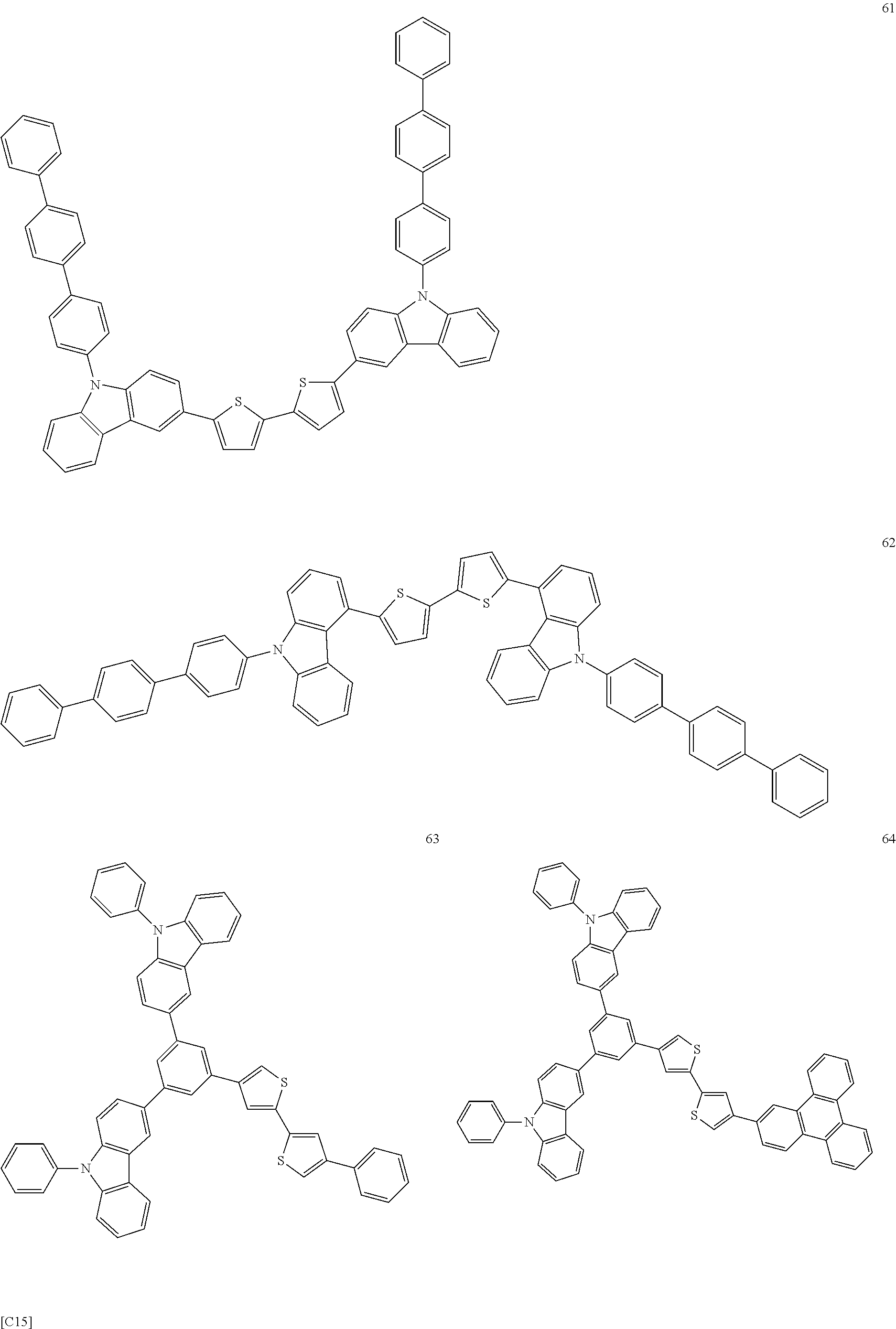

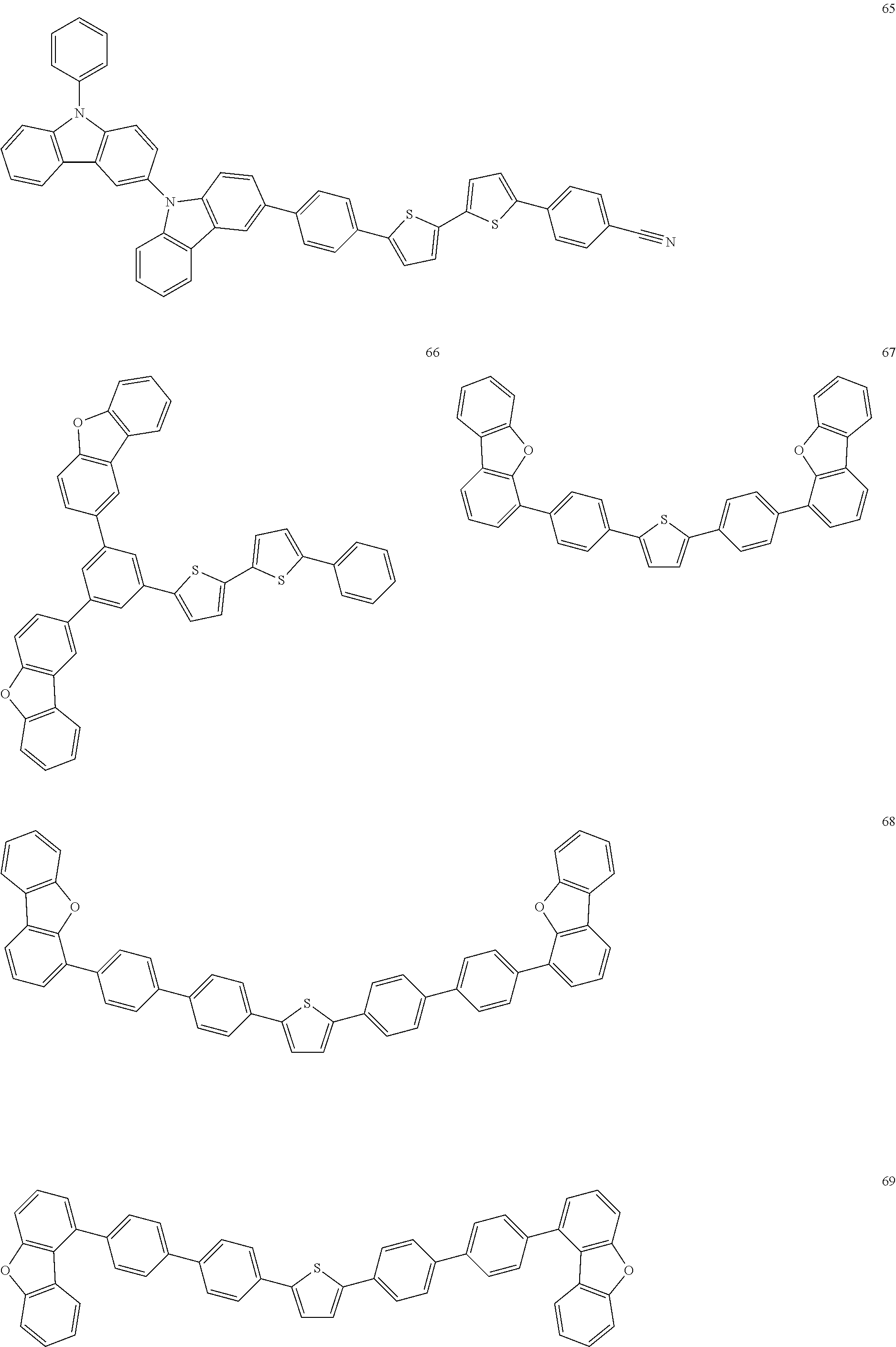

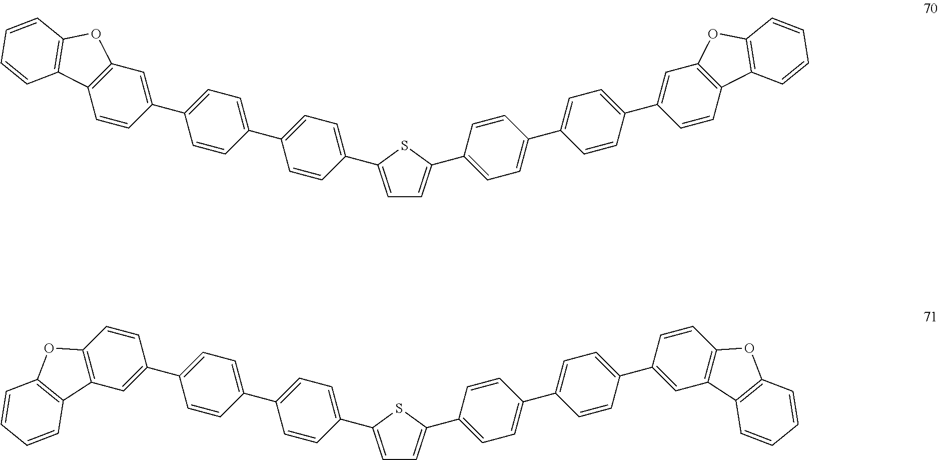

The material for a photoelectric conversion device of the present invention can be obtained by: synthesis by methods of various organic synthetic reactions established in the field of the organic synthetic chemistry including coupling reactions such as Suzuki coupling, Stille coupling, Grignard coupling, Ullmann coupling, Buchwald-Hartwig reaction, and Heck reaction, using commercially available reagents as raw materials; and then purification by using a known method such as recrystallization, column chromatography, and sublimation and purification. The method is not limited to this method.

The material for a photoelectric conversion device of the present invention preferably has an energy level of highest occupied molecular orbital (HOMO) obtained by structural optimization calculation with a density functional calculation B3LYP/6-31G (D) of −4.5 eV or lower, more preferably within a range of −4.8 eV to −6.0 eV.

An energy level of lowest unoccupied molecular orbital (LUMO) obtained by the above structural optimization calculation is preferably −2.5 eV or higher, more preferably within a range of −2.5 eV to −1.3 eV.

In the material for a photoelectric conversion device of the present invention, a difference (absolute value) between the HOMO energy level and the LUMO energy level is preferably within a range of 2.0 to 5.0 eV, and more preferably within a range of 2.5 to 4.0 eV.

The material for a photoelectric conversion device of the present invention preferably has a hole mobility of 1×10⁻⁶ cm²/Vs to 1 cm²/Vs, more preferably has a hole mobility of 1×10⁻⁵ cm²/Vs to 1×10⁻¹ cm²/Vs. The hole mobility can be evaluated by known methods such as a method with a FET-type transistor device, a method with a time-of-flight method, and an SCLC method.

The material for a photoelectric conversion device of the present invention is preferably amorphous. The amorphousness can be confirmed by various methods, and can be confirmed by, for example, detecting no peak in an XRD method or by detecting no endothermic peak in a DSC method.

Next, a photoelectric conversion device for imaging using the material for a photoelectric conversion device of the present invention will be described, but a structure of the photoelectric conversion device for imaging of the present invention is not limited thereto. The description will be made with reference to the drawings.

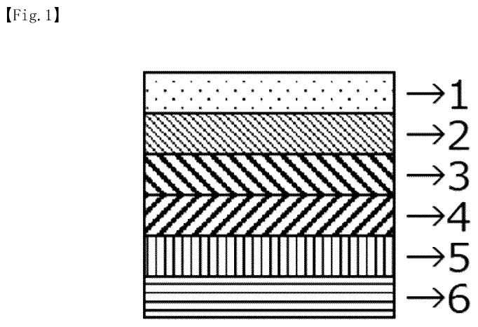

FIG. 1 is a sectional view schematically illustrating a structure of the photoelectric conversion device for imaging using the material for the photoelectric conversion device for imaging of the present invention. In FIG. 1, 1 represents a substrate, 2 represents an electrode, 3 represents an electron blocking layer, 4 represents a photoelectric conversion layer, 5 represents a hole blocking layer, and 6 represents an electrode. The photoelectric conversion device is not limited to the structure in FIG. 1, and adding or omitting a layer can be made as necessary.

Hereinafter, each member and each layer of the photoelectric conversion device of the present invention will be described.

### —Substrate—

The photoelectric conversion device is preferably supported on a substrate. This substrate is not particularly limited, and a substrate composed of glass, a transparent plastic, quartz, or the like may be used, for example.

### —Electrode—

An electrode used for the photoelectric conversion device for imaging of the present invention has a function of trapping a hole and an electron generated in the photoelectric conversion layer. A function to let light enter the photoelectric conversion layer is also required. Thus, at least one of two electrodes is desirably transparent or semi-transparent. A material used for the electrode is not particularly limited as long as it has conductivity, and examples thereof include: conductive transparent materials, such as ITO, IZO, SnO₂, ATO (antimony-doped tin oxide), ZnO, AZO (Al-doped zinc oxide), GZO (gallium-doped zinc oxide), TiO₂, and FTO; metals, such as gold, silver, platinum, chromium, aluminum, iron, cobalt, nickel, and tungsten; inorganic conductive substances, such as copper iodide and copper sulfide; and conductive polymers, such as polythiophene, polypyrrole, and polyaniline. A plurality of these materials may be mixed to use as necessary. In addition, two or more layers thereof may be stacked.

### —Photoelectric Conversion Layer—

The photoelectric conversion layer is a layer in which a hole and an electrode are generated by charge separation of an exciton generated by the incident light. The photoelectric conversion layer may be formed with a single photoelectric converting material, or may be formed by combination with a P-type organic semiconductor material being a hole transport material and an N-type organic semiconductor material being an electron transport material. Two or more kinds of the P-type organic semiconductor may be used, and two or more kinds of the N-type organic semiconductor may be used. One or more kinds of these P-type organic semiconductor and/or N-type organic semiconductor desirably use a dye material having a function of absorbing light with a desired wavelength in the visible region. As the P-type organic semiconductor material being the hole transport material, the material for a photoelectric conversion device of the present invention can be used.

The P-type organic semiconductor material may be any material having a hole transportability. The material for a photoelectric conversion device of the present invention is preferably used, but another P-type organic semiconductor material may be used. Two or more types of the compound represented by the general formula (1) or the general formula (1T) (the material for a photoelectric conversion device of the present invention) may be mixed to use. Furthermore, the above compound and another P-type organic semiconductor material may be mixed to use.

The another P-type organic semiconductor material may be any material having the hole transportability, and examples thereof include: aromatic compounds such as a naphthalene derivative, an anthracene derivative, a phenanthrene derivative, a pyrene derivative, a chrysene derivative, a naphthacene derivative, a triphenylene derivative, a perylene derivative, a fluoranthene derivative, a fluorene derivative, a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, a carbazole derivative, and indolocarbazole; an aromatic amine derivative, a styrylamine derivative, a benzidine derivative, a porphyrin derivative, a phthalocyanine derivative, and a quinacridone derivative.

As the P-type organic semiconductor material, a polymer material may be used. Examples of the polymer P-type organic semiconductor material include a polyphenylene-vinylene derivative, a polyparaphenylene derivative, a polyfluorene derivative, a polyvinylcarbazole derivative, and a polythiophene derivative. Two or more kinds selected from the compounds represented by the general formula (1) or the general formula (1T) of the present invention, the P-type organic semiconductor material, and the polymer P-type organic semiconductor material may be mixed to use.

The N-type organic semiconductor material may be any material having the electron transportability, and examples thereof include naphthalenetetracarboxylic diimide and perylenetetracarboxylic diimide, fullerenes (fullerene derivative), and azole derivatives such as imidazole, thiazole, thiadiazole, oxazole, oxadiazole, and triazole. Two or more kinds of materials selected from the N-type organic semiconductor materials may be mixed to use.

### —Electron Blocking Layer—

The electron blocking layer is provided in order to inhibit a dark current generated by injecting an electron from one electrode into the photoelectric conversion layer when a bias voltage is applied between the two electrodes. The electron blocking layer also has a function of hole transportation for transporting a hole generated by charge separation in the photoelectric conversion layer toward the electrode. A single layer or multiple layers of the electron blocking layer can be disposed as necessary. For the electron blocking layer, a P-type organic semiconductor material being the hole transport material can be used. The P-type organic semiconductor material may be any material having the hole transportability. Although the compound represented by the general formula (1) or the general formula (1T) is preferably used, another P-type organic semiconductor material may be used. In addition, the compound represented by the general formula (1) or the general formula (1T) and the other P-type organic semiconductor material as noted above or a polymer P-type organic semiconductor material may be mixed to use.

### —Hole Blocking Layer—

The hole blocking layer is provided in order to inhibit a dark current generated by injecting a hole from one electrode into the photoelectric conversion layer when a bias voltage is applied between the two electrodes. The hole blocking layer also has a function of electron transportation for transporting an electron generated by charge separation in the photoelectric conversion layer toward the electrode. A single layer or multiple layers of the hole blocking layer can be disposed as necessary. For the hole blocking layer, the N-type organic semiconductor material having the electron transportability can be used. The N-type organic semiconductor material may be any material having the electron transportability, and examples thereof include: polycyclic aromatic multivalent carboxylic anhydride or imidized products thereof, such as naphthalenetetracarboxylic diimide and perylenetetracarboxylic diimide; fullerenes (fullerene derivative), such as C60 and C70; azole derivatives, such as imidazole, thiazole, thiadiazole, oxazole, oxadiazole, and triazole; a tris(8-quinolinolate)aluminum (III) derivative, a phosphine oxide derivative, a nitro-substituted fluorene derivative, a diphenylquinone derivative, a thiopyran dioxide derivative, a carbodiimide, a fluorenylidene methane derivative, an anthraquinodimethane derivative and an anthrone derivative, a bipyridine derivative, a quinoline derivative, and an indolocarbazole derivative. Two or more kinds of materials selected from the N-type organic semiconductor materials may be mixed to use.

Hydrogen in the material of the present invention may be deuterium. That is, a part or all of hydrogens on the aromatic ring, in addition to hydrogens on the aromatic ring in the general formula (1) or the general formula (1T) and including the formulae (2) to (6), (6a) to (6d) and the substituent, may be a deuterium.

Furthermore, a part or all of hydrogens in a compound used as the N-type organic semiconductor material and the P-type organic semiconductor material may be deuterium.

A method for producing a film of each layer in producing the photoelectric conversion device for imaging of the present invention is not particularly limited. The photoelectric conversion device may be produced by any one of dry process and wet process.

The organic layer containing the material for a photoelectric conversion device of the present invention may be a plurality of layers as necessary.

### EXAMPLES

Hereinafter, the present invention will be described in more detail with Examples, but the present invention is not limited to these Examples.

### Calculation Example

### Calculation of HOMO and LUMO Values

Calculated were HOMO and LUMO for the compounds 1, 5, 6, 9, 18, 20, 25, 35, and 68, which were specific examples of the material represented by the aforementioned general formula (1) or the general formula (1T). The calculation was performed by using a density functional theory (DFT), using Gaussian as a calculation program, and with structural optimization calculation of a density functional calculation B3LYP/6-31G (d). Table 1 shows the results. It can be mentioned that any of the materials of the present invention has preferable HOMO and LUMO values.

As comparison, HOMO and LUMO for the compounds H1, H2, and H3 were calculated by the same method as the above method. Table 1 shows the results.

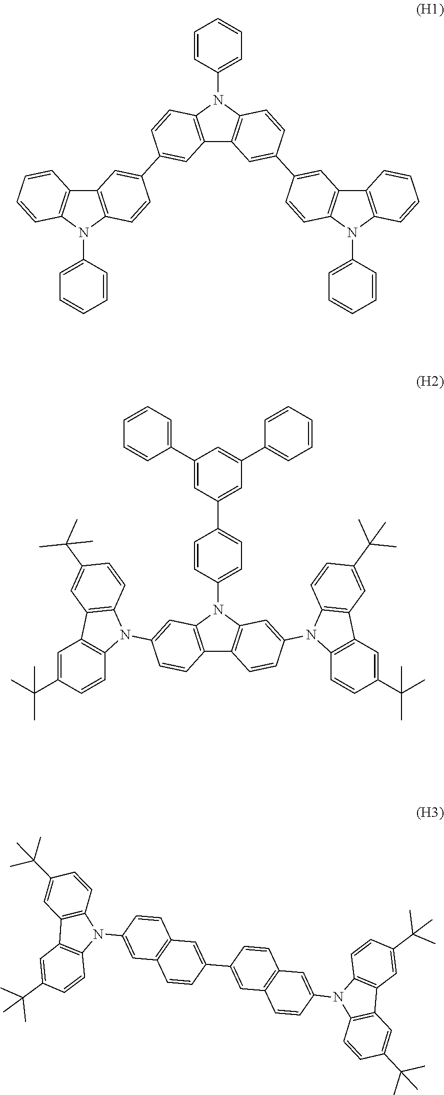

TABLE 1

| Compound | HOMO[eV] | LUMO[eV] |
|---|---|---|
| 1 | −4.9 | −1.7 |
| 5 | −4.9 | −1.8 |
| 6 | −4.9 | −1.8 |
| 9 | −5.0 | −1.7 |
| 18 | −5.1 | −1.9 |
| 20 | −4.9 | −2.0 |
| 25 | −5.0 | −1.5 |
| 35 | −4.8 | −1.6 |
| 68 | −5.2 | −1.6 |
| H1 | −4.9 | −0.7 |
| H2 | −5.0 | −1.3 |
| H3 | −5.3 | −1.3 |

Synthesis examples of the compounds 1, 5, 6, 9, 18, 20, 25, 35, and 68 will be described below as representative examples. The other compounds were also synthesized in the similar method.

### Synthesis Example 1 (Synthesis of Compound 1)

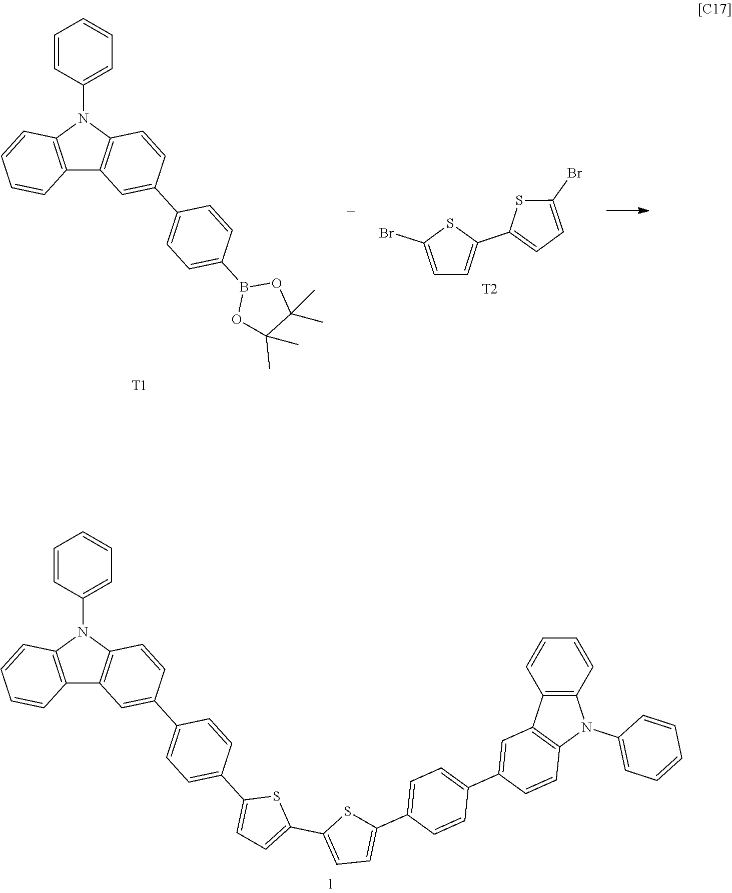

Into a three-necked 500-ml flask with degassed and nitrogen-replenished, T1 (10.2 mmol), T2 (5.1 mmol), tetrakis(triphenylphosphine) palladium (0) (0.5 mmol), and potassium carbonate (50.4 mmol) were added, and 100 ml of toluene, 25 ml of ethanol, and 25 ml of water were added thereinto, and then the mixture was stirred at 100° C. for 4 hours. The mixture was once cooled to a room temperature, then 200 ml of water was added, the mixture was transferred into a separatory funnel and separation into an organic layer and an aqueous layer was performed. The organic layer was washed three times with 200 ml of water, and then the obtained organic layer was concentrated under a reduced pressure. The obtained residue was reprecipitated with xylene to obtain the compound 1 (pale yellow solid). The yield was 42%. The obtained solid was evaluated by the XRD method, but no peak was detected.

### Synthesis Example 2 (Synthesis of Compound 5)

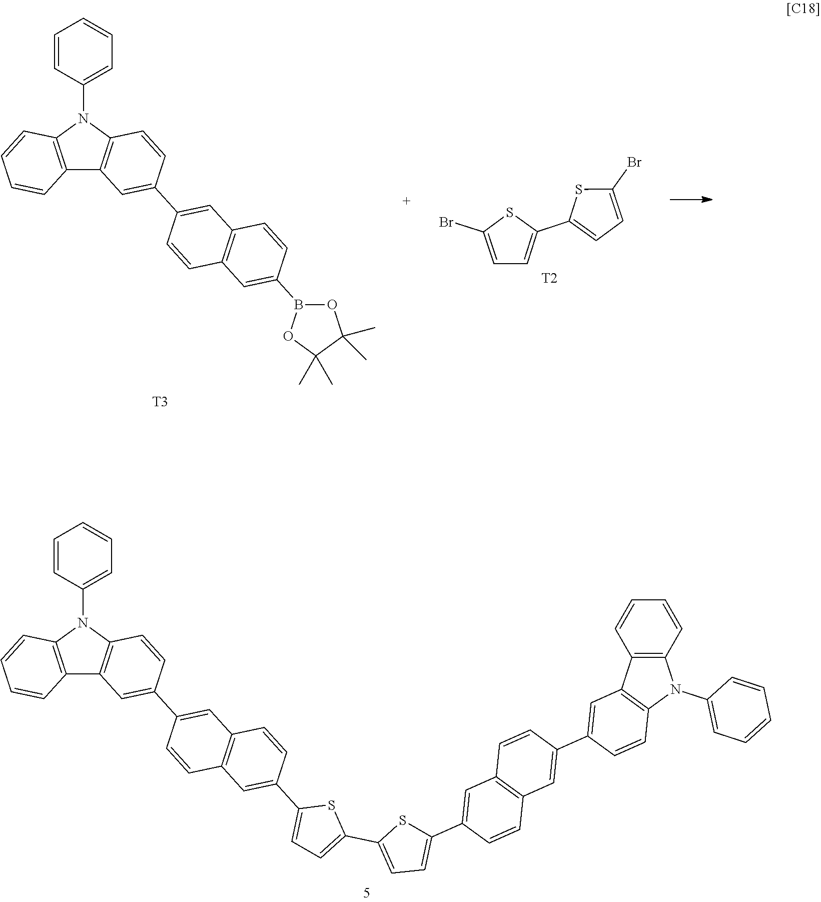

The compound 5 (pale yellow solid) was obtained in the same manner as in Synthesis Example 1 except that T1 was changed to T3. The yield was 49%. The obtained solid was evaluated by the XRD method, but no peak was detected.

### Synthesis Example 3 (Synthesis of Compound 6)

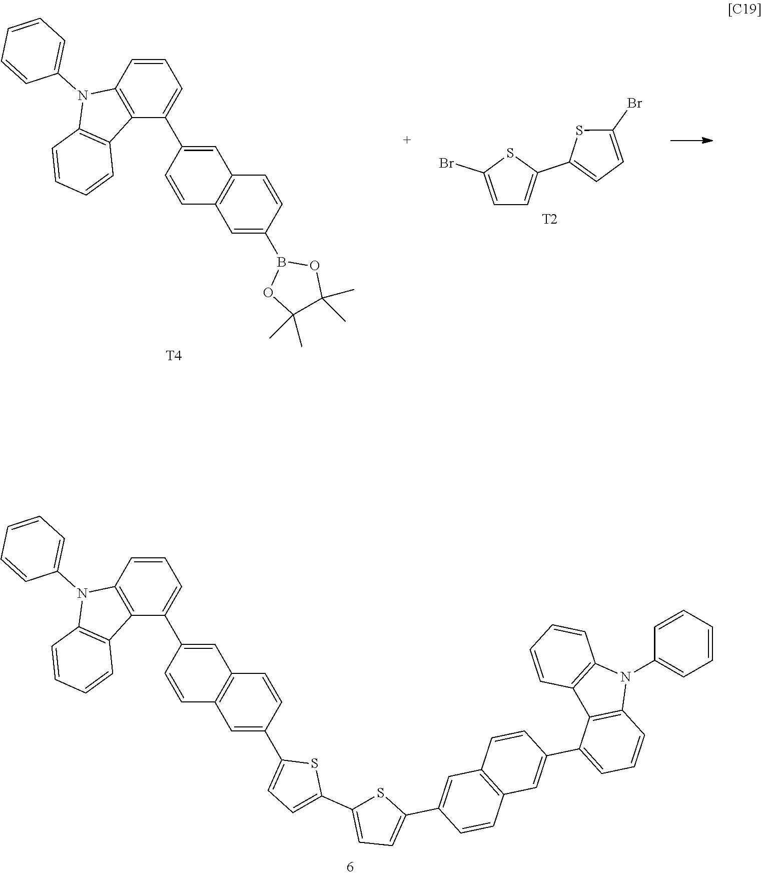

The compound 6 (pale yellow solid) was obtained in the same manner as in Synthesis Example 1 except that T1 was changed to T4. The yield was 49%. The obtained solid was evaluated by the XRD method, but no peak was detected.

### Synthesis Example 4 (Synthesis of Compound 9)

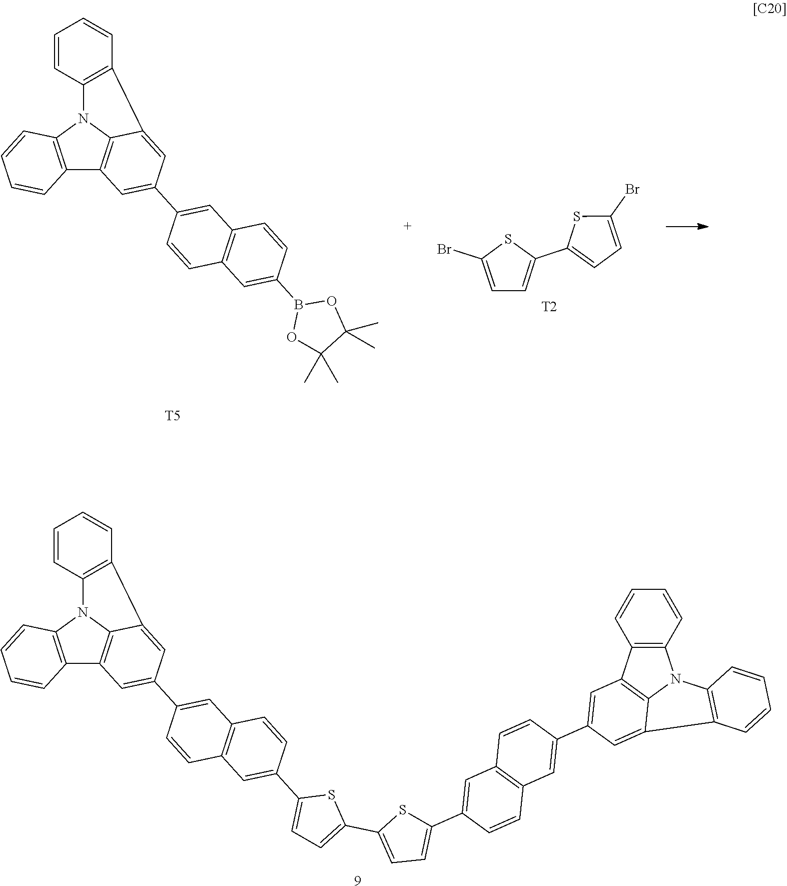

The compound 9 (pale yellow solid) was obtained in the same manner as in Synthesis Example 1 except that T1 was changed to T5. The yield was 55%. The obtained solid was evaluated by the XRD method, but no peak was detected.

### Synthesis Example 5 (Synthesis of Compound 18)

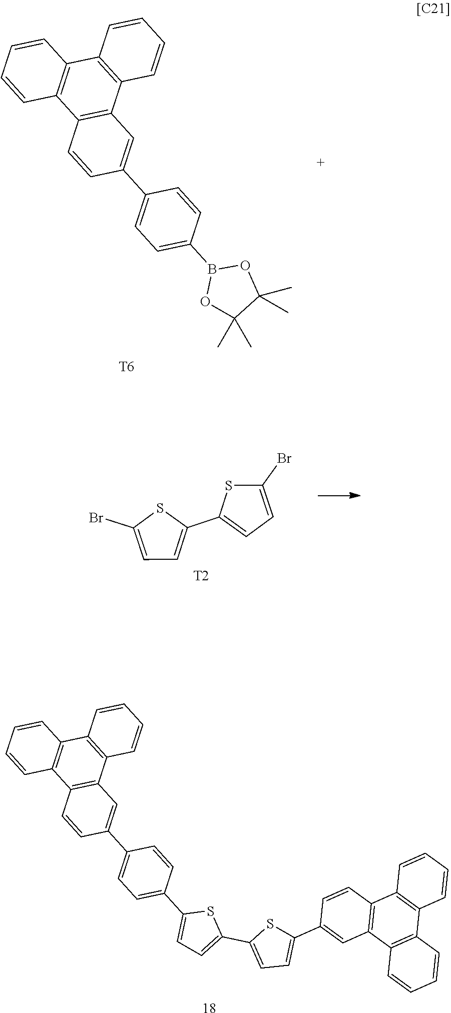

The compound 18 (pale yellow solid) was obtained in the same manner as in Synthesis Example 1 except that T1 was changed to T6. The yield was 17%.

### Synthesis Example 6 (Synthesis of Compound 20)

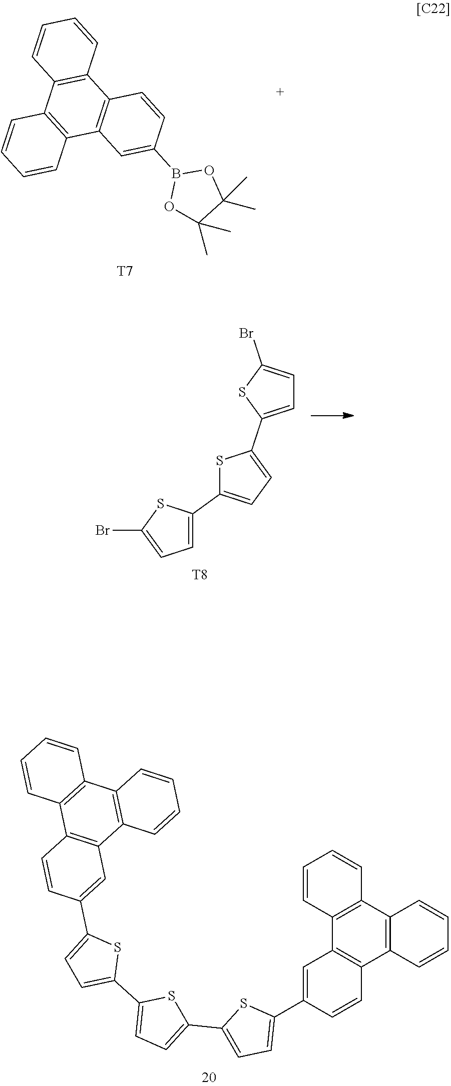

Into a three-necked 500-ml flask with degassed and nitrogen-replenished, T7 (24.6 mmol), T8 (9.8 mmol), tetrakis(triphenylphosphine) palladium (0) (0.3 mmol), and potassium carbonate (49.2 mmol) were added, and 100 ml of toluene, 40 ml of ethanol, and 40 ml of water were added thereinto, and then the mixture was stirred at 110° C. for 5 hours. The mixture was once cooled to a room temperature, then 200 ml of water was added, the mixture was transferred into a separatory funnel and separation into an organic layer and an aqueous layer was performed. The organic layer was washed three times with 200 ml of water, and then the obtained organic layer was concentrated under a reduced pressure. The obtained residue was reprecipitated with xylene to obtain the compound 20 (pale yellow solid). The yield was 33%.

### Synthesis Example 7 (Synthesis of Compound 25)

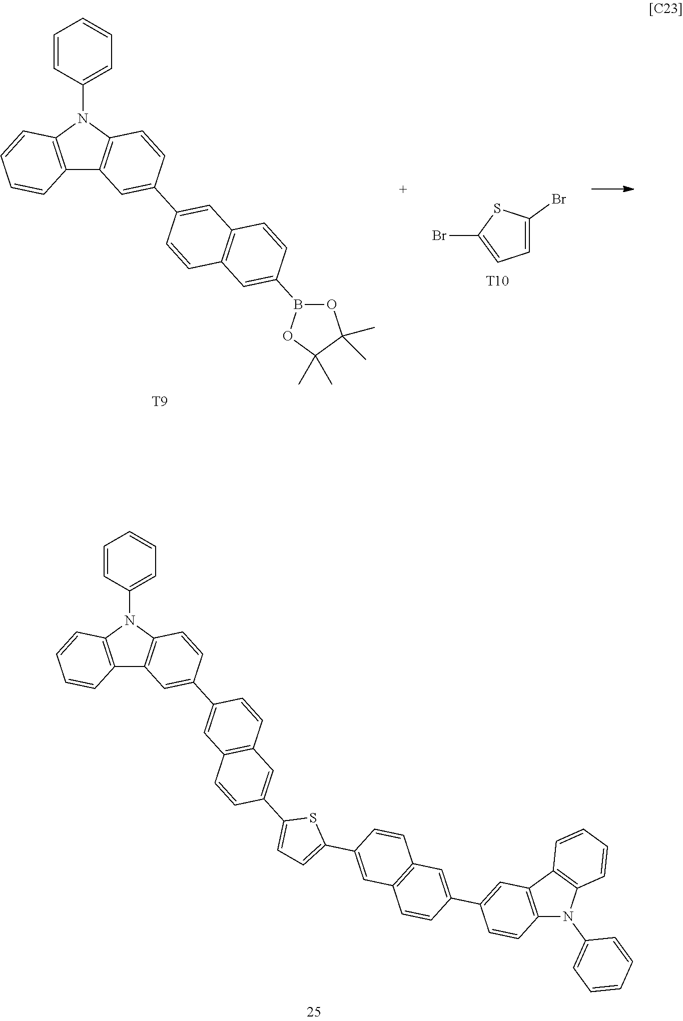

Into a three-necked 500-ml flask with degassed and nitrogen-replenished, T9 (30.7 mmol), T10 (14.1 mmol), tetrakis(triphenylphosphine) palladium (0) (0.8 mmol), and potassium carbonate (100.6 mmol) were added, and 200 ml of toluene, 50 ml of ethanol, and 50 ml of water were added thereinto, and then the mixture was stirred at 100° C. for 4 hours. The mixture was once cooled to a room temperature, then 200 ml of water was added, the mixture was transferred into a separatory funnel and separation into an organic layer and an aqueous layer was performed. The organic layer was washed three times with 200 ml of water, and then the obtained organic layer was concentrated under a reduced pressure. The obtained residue was reprecipitated with xylene to obtain the compound 25 (pale yellow solid). The yield was 57%. The obtained solid was evaluated by the XRD method, but no peak was detected.

### Synthesis Example 8 (Synthesis of Compound 35)

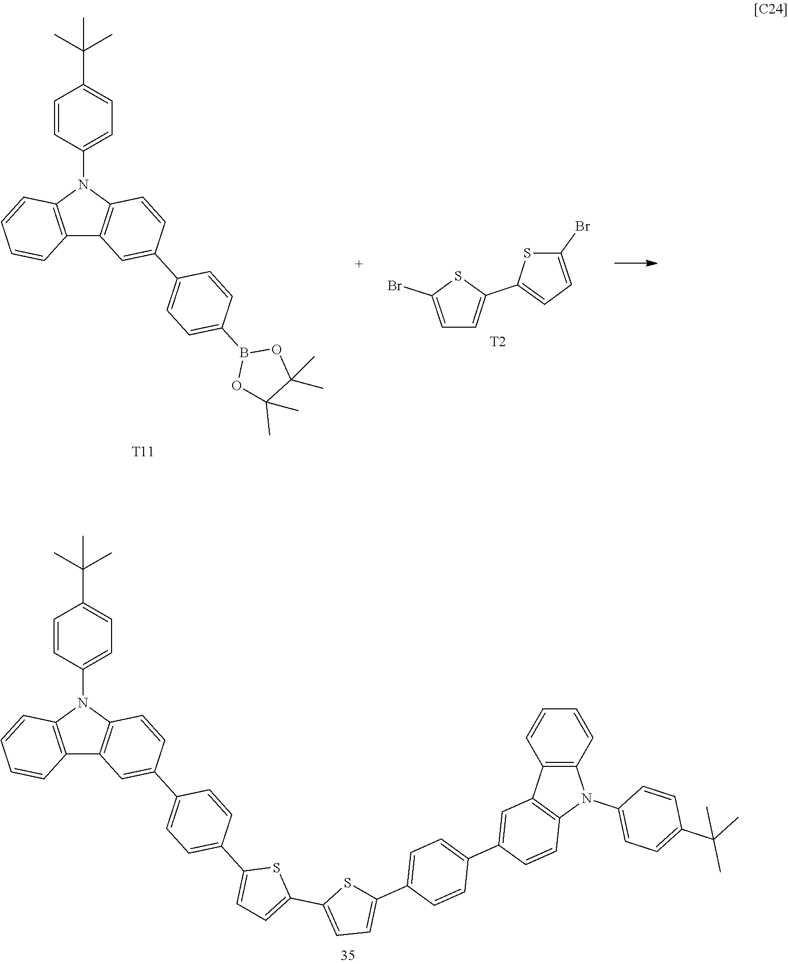

The compound 35 (pale yellow solid) was obtained in the same manner as in Synthesis Example 1 except that T1 was changed to T11. The yield was 60%. The obtained solid was evaluated by the XRD method, but no peak was detected.

### Synthesis Example 9 (Synthesis of Compound 68)

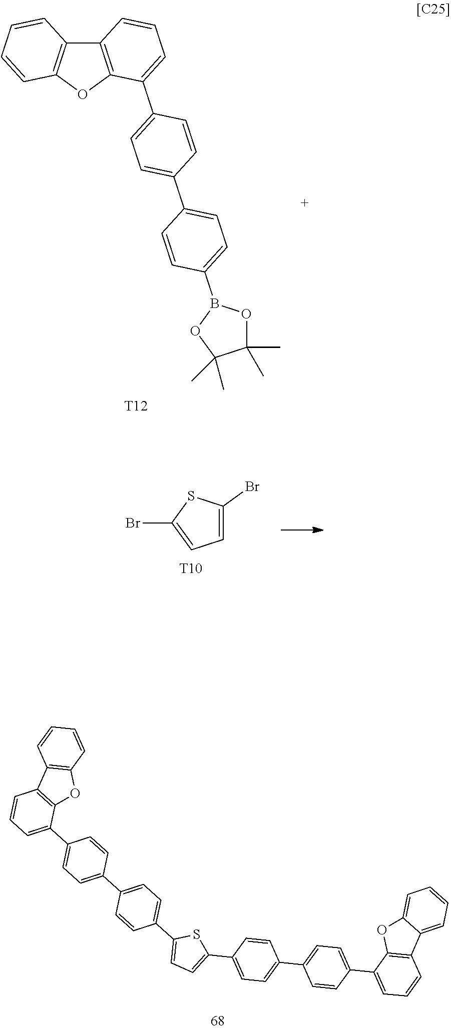

The compound 68 (pale yellow solid) was obtained in the same manner as in Synthesis Example 7 except that T9 was changed to T12. The yield was 74%. The obtained solid was evaluated by the XRD method, but no peak was detected.

### Measurement of Charge Mobility

On a glass substrate on which a transparent electrode composed of ITO with 110 nm in film thickness was formed, the compound 1 was produced to a film as an organic layer by a vacuum deposition method under a condition that a film thickness was approximately 3 μm. Subsequently, charge mobility was measured by a time-of-flight method using a device in which aluminum (Al) was formed with 70 nm in thickness as an electrode. The hole mobility was 3.1×10⁻⁴ cm²/Vs.

The hole mobility was measured by the same method as above except that compounds shown in Table 2 were used instead of the compound 1.

Table 2 shows the results.

TABLE 2

| Compound | Hole mobility [cm²/Vs] |
|---|---|
| 1 | 3.1 × 10⁻⁴ |
| 5 | 1.7 × 10⁻⁴ |
| 20 | 5.6 × 10⁻⁴ |
| 35 | 3.4 × 10⁻⁴ |
| 68 | 2.9 × 10⁻⁵ |
| H1 | 1.2 × 10⁻⁵ |
| H2 | 9.3 × 10⁻⁶ |

### Example 1

On an electrode composed of ITO with 70 nm in film thickness formed on a glass substrate, a 100-nm film of the compound 1 was formed with a vacuum degree of 4.0 x10-5 Pa as an electron blocking layer. Then, a 100-nm thin film of quinacridone was formed as a photoelectric conversion layer. Finally, a 70-nm aluminum film was formed as an electrode to produce a photoelectric conversion device.

A voltage of 2 V was applied between the ITO electrode and the aluminum electrode. In this time, a current in a dark place was 1.6×10⁻¹⁰ A/cm². When a voltage of 2 V was applied and the ITO electrode side was irradiated with light with an irradiation light wavelength of 500 nm and 1.6 μW/cm², a current was 1.6×10⁻⁷ A/cm². A contrast ratio was calculated to be 1.0×10³.

### Comparative Example 1

On an electrode, which was formed on a glass substrate, composed of ITO with 70 nm in film thickness, a 100-nm film of the compound H1 was formed with a vacuum degree of 4.0×10⁻⁵ Pa as an electron blocking layer. Then, a 100-nm film of quinacridone was formed as a photoelectric conversion layer. Finally, a 70-nm aluminum film was formed as an electrode to produce a photoelectric conversion device.

On this photoelectric conversion device, a current when a voltage of 2 V was applied in a dark place and a current with light irradiation were measured in the same manner as in Example 1. The current in a dark place was 5.6×10⁻⁹ A/cm², and the current with light irradiation was 1.2×10⁻⁷ A/cm². A contrast ratio was calculated to be 0.21×10² A/cm².

### Example 2

On an electrode composed of ITO with 70 nm in film thickness and formed on a glass substrate, a 10-nm film of the compound 1 was formed with a vacuum degree of 4.0×10⁻⁵ Pa as an electron blocking layer. Then, 2Ph-BTBT, F6-SubPc-OC6F5, and fullerene (C60) were co-deposited at a deposition rate ratio of 4:4:2 with 200 nm to form a film as a photoelectric conversion layer. Subsequently, 10-nm of dpy-NDI was deposited to form a hole blocking layer. Finally, an aluminum film was formed with 70 nm in thickness as an electrode to produce a photoelectric conversion device. A current in a dark place (dark current) was 4.2×10⁻¹⁰ A/cm² with the electrodes of ITO and aluminum and with applying a voltage of 2.6 V. When a voltage of 2.6 V was applied and the ITO electrode side was irradiated with light with an LED adjusted to be an irradiation light wavelength of 500 nm and 1.6 μW from a height of 10 cm, a current (bright current) was 2.9×10⁻⁷ A/cm². A contrast ratio was 6.9×10² with applying a voltage of 2.6 V. Table 3 shows the results.

### Examples 3 to 9

Photoelectric conversion devices were produced in the same manner as in Example 2 except that compounds shown in Table 3 were used as the electron blocking layer.

### Comparative Examples 2 and 3

Photoelectric conversion devices were produced in the same manner as in Example 2 except that compounds shown in Table 3 were used as the electron blocking layer.

Table 3 shows the results in Examples 2 to 9 and Comparative Examples 2 and 3.

The compounds used in Examples and Comparative Examples are shown below.

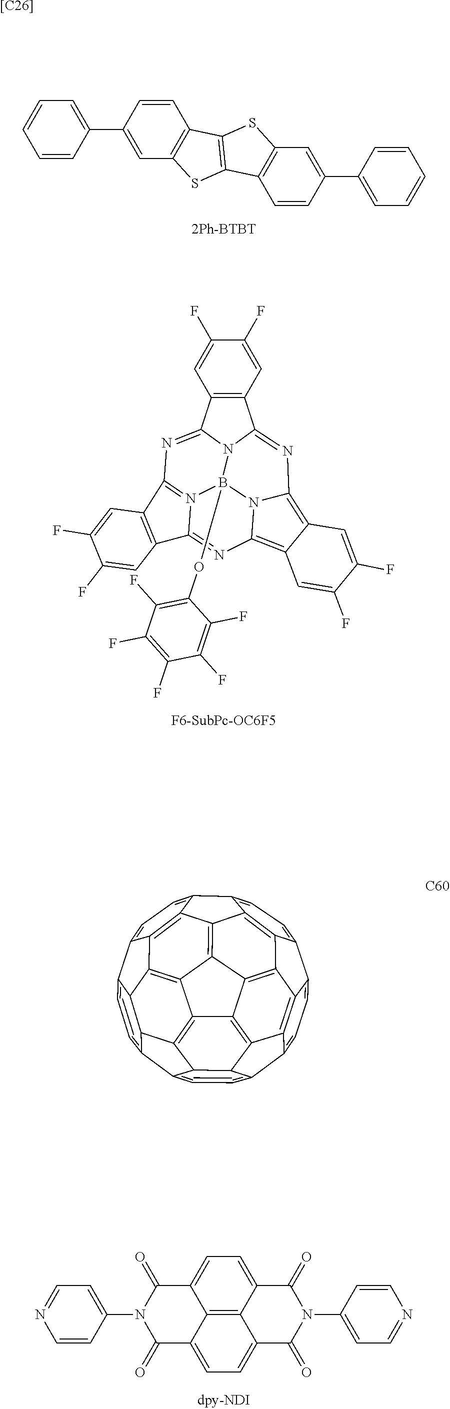

TABLE 3

| | Compound | | Current value | |
|---|---|---|---|---|
| | of electron | Current value | with light | |
| | blocking | in dark place | irradiation | Contrast |
| | layer | [A/cm²] | [A/cm²] | ratio |
| Example 2 | 1 | 4.2 × 10⁻¹⁰ | 2.9 × 10⁻⁷ | 6.9 × 10² |
| Example 3 | 5 | 4.4 × 10⁻¹⁰ | 3.1 × 10⁻⁷ | 7.0 × 10² |
| Example 4 | 6 | 4.1 × 10⁻¹⁰ | 2.7 × 10⁻⁷ | 6.6 × 10² |
| Example 5 | 9 | 4.0 × 10⁻¹⁰ | 2.8 × 10⁻⁷ | 7.0 × 10² |
| Example 6 | 20 | 4.2 × 10⁻¹⁰ | 2.9 × 10⁻⁷ | 6.9 × 10² |
| Example 7 | 25 | 3.9 × 10⁻¹⁰ | 2.7 × 10⁻⁷ | 6.9 × 10² |
| Example 8 | 35 | 3.9 × 10⁻¹⁰ | 2.6 × 10⁻⁷ | 6.7 × 10² |
| Example 9 | 68 | 3.6 × 10⁻¹⁰ | 2.4 × 10⁻⁷ | 6.7 × 10² |
| Comparative | H1 | 7.7 × 10⁻¹⁰ | 2.4 × 10⁻⁷ | 3.1 × 10² |
| Example 2 | | | | |
| Comparative | H2 | 6.1 × 10⁻¹⁰ | 2.0 × 10⁻⁷ | 3.3 × 10² |
| Example 3 | | | | |

It is found from the results in Table 3 that the photoelectric conversion devices using the compound of the present invention exhibit a low dark current value and a high contrast ratio.

### INDUSTRIAL APPLICABILITY

The material for a photoelectric conversion device for imaging of the present invention can achieve appropriate move of the hole and the electron in the photoelectric conversion device, and consequently enables to reduce a leakage current generated by applying a bias voltage during the conversion of light into electric energy. As a result, a photoelectric conversion device that achieves a low dark current value and a high contrast ratio can be obtained. The material of the present invention is useful as a material for a photoelectric conversion device for a photoelectric-converting film-stacked imaging device.

### REFERENCE SIGNS LIST

1 Substrate, 2 Electrode, 3 Electron blocking layer, 4 Photoelectric conversion layer, 5 Hole blocking layer, 6 Electrode.

1. A material for a photoelectric conversion device for imaging, the material comprising a thiophene derivative represented by the following general formula (1): 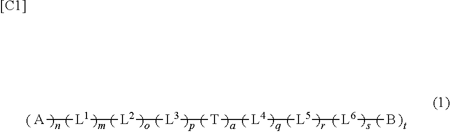
wherein A and B each independently represent hydrogen, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to four of these aromatic groups are linked, and at least one of A or B has a condensed-ring structure having 12 or more carbon atoms represented by the following general formula (2) or (3);
L¹, L², L³, L⁴, L⁵, and L⁶ each independently represent a substituted or unsubstituted aromatic hydrocarbon group having 6 to 30 carbon atoms, or a substituted or unsubstituted aromatic heterocyclic group having 3 to 18 carbon atoms;
T represents a substituted or unsubstituted thiophene ring; “a” represents the number of repetition and represents an integer of 1 to 4; “m”, “o”, “p”, “q”, “r”, and “s” represent the number of linkage and each independently represent an integer of 0 or 1; and “n” and “t” represent the number of substitution and each independently represent 1 or 2, 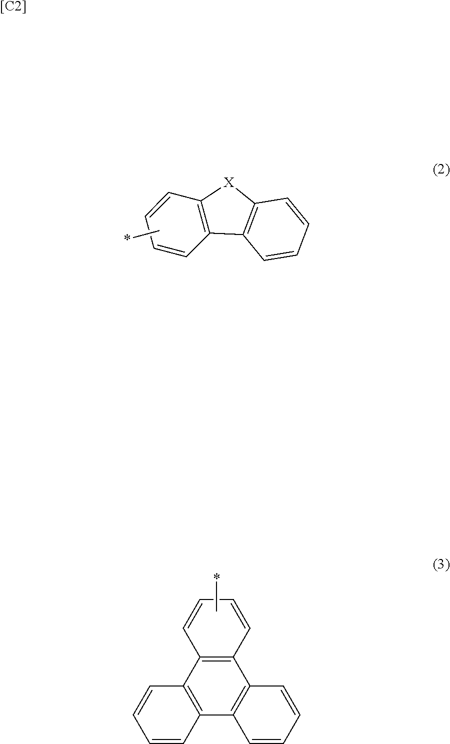
wherein “*” represents a bonding position at which the formula (2) or (3) is substituted, the condensed-ring structures represented by the formulae (2) and (3) optionally have a substituent, and the formula (2) is optionally condensed with the substituent to form a condensed ring;
X represents N—Ra, O, S, or C—(Rb)₂, Ra and Rb each independently represent a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to four of these aromatic groups are linked, and Ra and Rb are optionally bonded to the formula (2) to form a condensed ring.

2. The material for a photoelectric conversion device for imaging according to claim 1, wherein the general formula (1) is represented by the following general formula (1T): 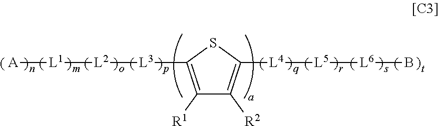
wherein A, B, L¹ to L⁶, “a”, and “n” to “t” are as defined for the general formula (1); R¹ and R² each independently represent hydrogen, a substituted or unsubstituted aromatic hydrocarbon group having 6 to 30 carbon atoms, or a substituted or unsubstituted aromatic heterocyclic group having 3 to 18 carbon atoms.

3. The material for a photoelectric conversion device for imaging according to claim 1, wherein in the general formula (1), at least one of A or B is represented by the general formula (2), and X in the formula (2) represents N—Ra or O.

4. The material for a photoelectric conversion device for imaging according to claim 3, wherein in the general formula (1), at least one of A or B is represented by the following general formula (4): 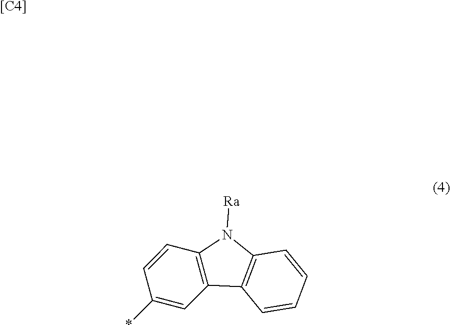
wherein “*” is as defined for the formula (2); the formula (4) optionally has a substituent; Ra is as defined for the formula (2), and optionally further forms a condensed ring with the formula (4) itself or the substituent in the formula (4); and the formula (4) is optionally condensed with the substituent in the formula (4) itself to form a condensed ring.

5. The material for a photoelectric conversion device for imaging according to claim 4, wherein in the general formula (1), both A and B are represented by the general formula (4).

6. The material for a photoelectric conversion device for imaging according to claim 1, wherein in the general formula (1), at least one of A or B is represented by the following general formula (5): 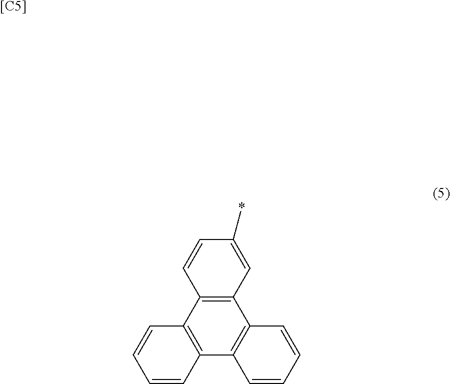
wherein “*” is as defined for the formula (3); and the formula (5) optionally has a substituent.

7. The material for a photoelectric conversion device for imaging according to claim 6, wherein in the general formula (1), both A and B are represented by the general formula (5).

8. The material for a photoelectric conversion device for imaging according to claim 1, wherein in the general formula (1), at least one of L¹, L², L³, L⁴, L⁵, or L⁶ is represented by the following general formula (6): 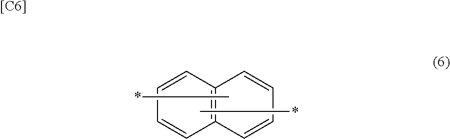
wherein “*” represents a bonding position at which the formula (6) is substituted with A, B, L¹, L², L³, L⁴, L⁵, L⁶, or T; and the formula (6) optionally has a substituent.

9. The material for a photoelectric conversion device for imaging according to claim 8, wherein in the general formula (1), at least one of L¹, L², L³, L⁴, L⁵, or L⁶ is represented by the following general formula (6a) to (6d): 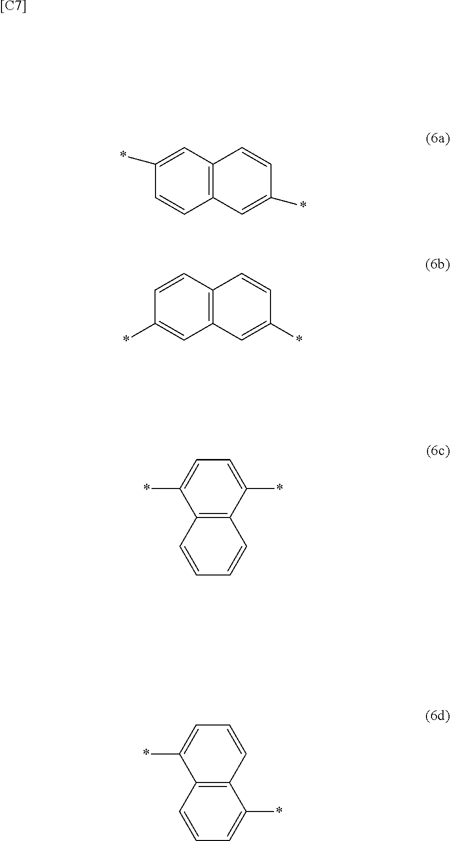
wherein “*” is as defined for the formula (6); and the formulae (6a) to (6d) optionally have a substituent.

10. The material for a photoelectric conversion device according to claim 1, wherein in the general formula (1), “a” represents 1 to 3; and p=q=0.

11. The material for a photoelectric conversion device according to claim 1, wherein an energy level of highest occupied molecular orbital (HOMO) obtained by structural optimization calculation with a density functional calculation B3LYP/6-31G (d) is −4.5 eV or lower.

12. The material for a photoelectric conversion device according to claim 1, wherein an energy level of lowest unoccupied molecular orbital (LUMO) obtained by structural optimization calculation with a density functional calculation B3LYP/6-31G (d) is −2.5 eV or higher.

13. The material for a photoelectric conversion device according to claim 1, wherein the material has a hole mobility of 1×10⁻⁶ cm²/Vs or more.

14. The material for a photoelectric conversion device according to claim 1, wherein the material is amorphous.

15. The material for a photoelectric conversion device according to claim 1, wherein the material is used as a hole transport material of a photoelectric conversion device for imaging.

16. A photoelectric conversion device for imaging, comprising a photoelectric conversion layer and an electron blocking layer between two electrodes, wherein at least one layer of the photoelectric conversion layer and the electron blocking layer contains the material for a photoelectric conversion device according to claim 1.

17. The photoelectric conversion device for imaging according to claim 16, wherein the electron blocking layer contains the material for a photoelectric conversion device.

18. The photoelectric conversion device for imaging according to claim 16, wherein the photoelectric conversion layer contains an electron transport material.

19. The photoelectric conversion device for imaging according to claim 16, wherein the electron blocking layer contains the material for a photoelectric conversion device, and the photoelectric conversion layer contains a fullerene derivative.
